(19) United States
(12) Patent Application Publication
(10) Pub. No.: US 2022/0387460 A1
(43) Pub. Date: Dec. 8, 2022

(54) LIPOSOME COMPOSITIONS AND METHODS OF TREATMENT TARGETED TO TUMOR ENDOTHELIUM

(71) Applicant: Washington University, St. Louis, MO (US)

(72) Inventors: Abdel Kareem Azab, St. Louis, MO (US); Cinzia Federico, St. Louis, MO (US)

(73) Assignee: Washington University, St. Louis, MO (US)

(21) Appl. No.: 17/776,186

(22) PCT Filed: Nov. 11, 2020
(86) PCT No.: PCT/US20/60004

§ 371 Date: May 11, 2022

Related U.S. Application Data
(60) Provisional application No. 62/933,720, filed on Nov. 11, 2019.

(51) Int. Cl. A61K 31/69; A61K 9/127; A61K 9/00; A61K 9/48; A61K 47/69; A61P 35/00

(52) U.S. Cl. CPC A61K 31/69; A61K 9/127; A61K 9/0019; A61K 9/4891; A61K 47/6911; A61P 35/00

(57) ABSTRACT

Compositions and methods of treatment for multiple myeloma (MM) are disclosed that include a liposome with a lipid bilayer shell enclosing a fluid-filled center, a targeting moiety coupled to the outer surface of the shell, a treatment compound disposed within the lipid bilayer shell or within the fluid-filled center, and an efficacy-enhancing compound disposed within the lipid bilayer shell or within the fluid-filled center. In some embodiments, the targeting moiety is PSGL-1, the proteasome-inhibiting compound is bortezomib, and the BMME-disrupting agent is a CXCR4 inhibitor or ROCK inhibitor.

### CROSS-REFERENCE TO RELATED APPLICATIONS

This application claims priority from U.S. Provisional Application Ser. No. 62/933,720 filed on Nov. 11, 2019, which is incorporated herein by reference in its entirety.

### STATEMENT REGARDING FEDERALLY SPONSORED RESEARCH OR DEVELOPMENT

This invention was made with government support under CA199092 awarded by the National Institutes of Health. The government has certain rights in the invention.

### FIELD OF THE DISCLOSURE

The present disclosure generally relates to compositions and methods of treatment targeted to tumor endothelium, and in particular, the present disclosure relates to compositions and methods for treating multiple myeloma.

### BACKGROUND OF THE DISCLOSURE

Multiple myeloma (MM), the second most common hematological malignancy, is characterized by the neoplastic transformation and growth of plasma cells within the bone marrow (BM). Treatment using therapeutic agents such as proteasome inhibitors (PIs) and immunomodulatory agents (IMiDs) has significantly improved the outcomes of MM patients. However, almost all MM patients become refractory to treatment and relapse due to de novo drug resistance.

Bone marrow microenvironment (BMME) has been implicated in the development of drug resistance in MM. The direct interaction of MM cells with the BM stroma, endothelial cells, and extracellular matrix, as well as the cytokines and chemokines present in the BM milieu, was shown to induce drug resistance in MM cells. Disruption of the interaction between MM cells and the BMME by the inhibition of CXCR4 or selectins was forwarded as one strategy for the sensitization of MM to therapy in vitro and in vivo. The interaction of MM cells with stromal and endothelial cells in the BMME was shown to be promoted through a cascade of cell signaling that involved Rho guanosine triphosphatases and inhibition of targets downstream of this cell signaling, such as Rho kinase (ROCK), resulted in the abrogation of the MM-BMME interaction. The combinational use of chemotherapeutic and BMME-disrupting agents such as bortezomib (BTZ) and a ROCK inhibitor, respectively, may represent a potential treatment of MM.

The effective use of chemotherapies in MM such as proteasome inhibitors (PIs) and immunomodulatory agents (IMiDs) may be accompanied by serious adverse effects. Treatment with PIs may be limited by neurotoxicity, especially in the peripheral nerves, which leads to painful sensory axonal neuropathy. Therefore, treatment strategies that specifically target MM cells to increase the efficacy of the treatment and that reduce off-tumor side effects are needed in the treatment of MM.

The emphasis in cancer treatment in general, and MM in particular, has been shifting from cytotoxic and non-specific chemotherapies to molecularly targeted and rationally designed therapies that exhibit greater efficacy and fewer side effects. Some therapeutic approaches have made use of nanoparticles for the treatment of MM. However, these nanoparticle treatments were typically non-targeted and were accompanied by considerable pharmacokinetic and pharmacodynamic disadvantages, including the lack of specificity and the dependency on the enhanced permeability and retention (EPR) effect.

Delivery of the proteasome inhibitor BTZ in a CD38-targeted cross-linked chitosan nanoparticle reduced the toxicity profile of BTZ in vivo. The anti-CD38 chitosan nanoparticles induced a low toxicity profile, which allowed the enhancement of proteasome-inhibitory activity and specificity of BTZ by endocytosis-mediated uptake of CD38. Although the targeted administration of BTZ in this manner was a promising therapy in MM, tumors subjected to this treatment eventually relapsed, most likely due to BMME-induced drug resistance.

### SUMMARY DESCRIPTION OF THE DISCLOSURE

In one aspect, a composition for treating multiple myeloma (MM) within a patient in need is disclosed. The composition includes a liposome with a lipid bilayer shell forming an outer surface and an inner surface enclosing a fluid-filled center, a targeting moiety coupled to the outer surface, a treatment compound disposed within the lipid bilayer shell or within the fluid-filled center, and an efficacy-enhancing compound disposed within the lipid bilayer shell or within the fluid-filled center. In some aspects, the targeting moiety includes PSGL-1. In other aspects, the treatment compound includes a proteasome-inhibiting compound. In additional aspects, the proteasome-inhibiting compound is bortezomib disposed within the lipid bilayer shell between the inner and outer surfaces. In other additional aspects, the bortezomib is disposed within the lipid bilayer shell at an encapsulation efficiency ranging from about 70% to about 80% or at an encapsulation efficiency of about 75%. In yet other additional aspects, the efficacy-enhancing compound is a BMME-disrupting agent selected from a CXCR4 inhibitor and a ROCK inhibitor. In additional aspects, the efficacy-enhancing compound is the ROCK inhibitor. In other additional aspects, the ROCK inhibitor includes Y27632 disposed within the fluid-filled center. In other aspects, the Y27632 is disposed within the fluid-filled center at an encapsulation efficiency ranging from about 40% to about 60%, or at an encapsulation efficiency of about 55%. In various additional aspects, the liposome has an average size ranging from about 10 nm to about 250 nm, from about 50 nm to about 200 nm, ranging from about 100 nm to about 200 nm, ranging from about 125 nm to about 175 nm, or has an average size of about 145 nm. In an additional aspect, the zeta potential of the liposomes is at least about 28 mV. In other additional aspects, the liposome further includes DPPC, Chol, DSPE-mPEG2000, and DSPE-PEG(2000)-succinyl, and any combination thereof. In yet other additional aspects, the liposome includes DPPC, Chol, DSPE-mPEG2000, and DSPE-PEG(2000)-succinyl at molar ratio of 6 DPPC:3 Chol: 0.5 DSPE-mPEG2000: 0.5 DSPE-PEG(2000)-succinyl is 6:3:0.5:0.5 (DPPC:Chol:DSPE-mPEG2000:DSPE-PEG(2000)-succinyl). In additional aspects, the fluid-filled center includes a hydrophilic fluid. In other additional aspects, the composition further includes a lipid carrier, wherein the liposomes are suspended within the liquid carrier. In additional aspects, the liposomes are suspended within the liquid carrier at a concentration of 2 mg of liposomes per mL of lipids.

In another aspect, a method of specifically delivering a therapeutic composition of PSGL-1 functionalized liposomes loaded with BTZ and Y27632 to tumor cells of a subject is disclosed. The method includes administering an effective amount of any of the therapeutic compositions described above to the subject. In some aspects, the therapeutic composition is administered by injection or infusion. In additional aspects, the therapeutic composition is injected or infused at a dose of about 2.5 mg/kg of BTZ and about 2.5 mg/kg of Y27632 within the same liposome.

Other objects and features will be in part apparent and in part pointed out hereinafter.

### DESCRIPTION OF THE DRAWINGS

Those of skill in the art will understand that the drawings described below are for illustrative purposes only. The drawings are not intended to limit the scope of the present teachings in any way.

FIG. 1A is a bar graph comparing P-selectin expression on the endothelial cells (ECs) of healthy and multiple myeloma (MM) subjects;

FIG. 1B is a bar graph comparing the expression of P-selectin on the bone marrow endothelium of healthy and MM-inoculated mice;

FIG. 1C is a confocal microscopy image showing MM (green) and MM-derived stroma (red) inside a patient-derived 3D tissue-engineered bone marrow (3DTEBM);

FIG. 1D is a confocal microscopy image showing ECs cultured for 24 hours on top of the 3DTEBM scaffold shown in FIG. 1C;

FIG. 1E is a bar graph comparing P-selectin expression of ECs when cultured alone or with MM cells in 2DTEBM or 3DTEBM;

FIG. 2A contains schematic illustrations of non-targeted and PSGL-1-targeted liposomes in accordance with one aspect of the disclosure;

FIG. 2B is a graph showing a time-series of immobilization of purified P-selectin onto a sensor chip via amine coupling;

FIG. 2C is a graph comparing a binding rate of P-selectin to non-targeted liposomes and PSGL-1-targeted liposomes using a BIAcore apparatus;

FIG. 2D is a graph comparing liposomal binding (MFI) of PSGL-1-targeted and non-targeted particles to ECs in vitro;

FIG. 2E is a graph comparing liposomal binding of PSGL-1-targeted and non-targeted particles to ECs in vitro;

FIG. 3A is a schematic illustration of a liposome loaded with a therapeutic compound (BTZ) and a BMME-disrupting agent (Y27632);

FIG. 3B is an HPLC calibration curve for BTZ;

FIG. 3C is a detection peak of BTZ obtained using HPLC;

FIG. 3D is an HPLC calibration curve for Y27632;

FIG. 3E is a detection peak of Y27632 obtained using HPLC;

FIG. 4A contains a series of images comparing immunoblotted adhesion signaling proteins from lysed MMs cultured with various treatments;

FIG. 4B contains a series of images comparing immunoblotted adhesion signaling proteins from lysed HUVECs cultured with various treatments;

FIG. 4C is a bar graph comparing trans-endothelial migration of MM cells cultured in vitro under various conditions: without the chemokine SDF-1, with SDF-1, and with SDF-1 in combination with free or liposomal Y27632;

FIG. 4D is a bar graph comparing percentages of MM cells circulating in the peripheral blood following in vivo administration of free Y27632, non-targeted liposomal Y27632, and PSGL-1-targeted liposomal Y27632;

FIG. 5A is a series of images comparing immunoblotted molecules from lysed MMs cultured with various treatments related to apoptosis (cPARP, p21, cCasp3, and cCasp9), cell cycle (pRB), and survival (pAKT, pS6R, and pERK);

FIG. 5B is a bar graph comparing the viability of MM cells following incubation with increasing concentrations of free or liposomal BTZ;

FIG. 5C is a bar graph comparing the viability of ECs following incubation with increasing concentrations of free or liposomal BTZ;

FIG. 6A is a graph comparing MM burden of mice treated with free forms of Y27632, BTZ, and Y27632+BTZ;

FIG. 6B is a graph comparing MM burden of mice treated with non-targeted liposomal forms of Y27632, BTZ, and Y27632+BTZ;

FIG. 6C is a graph comparing MM burden of mice treated with PSGL-1-targeted liposomal forms of Y27632, BTZ, and Y27632+BTZ;

FIG. 6D is a graph comparing the survival of mice treated with free forms of Y27632, BTZ, and Y27632+BTZ;

FIG. 6E is a graph comparing the survival of mice treated with non-targeted liposomal forms of Y27632, BTZ, and Y27632+BTZ;

FIG. 6F is a graph comparing the survival of mice treated with PSGL-1-targeted liposomal forms of Y27632, BTZ, and Y27632+BTZ;

FIG. 6G is a bar graph comparing weight changes of mice treated with free, non-targeted liposomal, and PSGL-1-targeted liposomal administration forms;

FIG. 6H contains a series of mouse photographic images summarizing hair loss experienced in vivo for BTZ and combination treatments (free, non-targeted, and PSGL-1-targeted administration forms);

FIG. 7 is a schematic illustration of a patient-derived 3D tissue-engineered bone marrow (3DTEBM);

FIG. 8A is a graph summarizing the tumor burden over a 28-day course of vehicle treatment using free, non-targeted liposome, and PSGL-1-targeted liposome administration;

FIG. 8B is a graph summarizing the tumor burden over a 28-day course of Y27632 treatment using free, non-targeted liposome, and PSGL-1-targeted liposome administration;

FIG. 8C is a graph summarizing the tumor burden over a 28-day course of BTZ treatment using free, non-targeted liposome, and PSGL-1-targeted liposome administration;

FIG. 8D is a graph summarizing the tumor burden over a 28-day course of combination (Y27632+BTZ) treatment using free, non-targeted liposome, and PSGL-1-targeted liposome administration;

FIG. 9A is a graph summarizing survival over a 28-day course of vehicle treatment using free, non-targeted liposome, and PSGL-1-targeted liposome administration;

FIG. 9B is a graph summarizing survival over a 28-day course of Y27632 treatment using free, non-targeted liposome, and PSGL-1-targeted liposome administration;

FIG. 9C is a graph summarizing survival over a 28-day course of BTZ treatment using free, non-targeted liposome, and PSGL-1-targeted liposome administration; and

FIG. 9D is a graph summarizing survival over a 28-day course of combination (Y27632+BTZ) treatment using free, non-targeted liposome, and PSGL-1-targeted liposome administration

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION OF THE DISCLOSURE

Drug resistance and dose-limiting toxicities remain significant barriers to the treatment of multiple myeloma (MM) and cancer in general. Without being limited to any particular theory, the bone marrow microenvironment (BMME) is thought to play a role in the development of drug resistance in MM. Drug delivery with targeted nanoparticles has been shown to achieve better specificity and efficacy, as well as to reduce toxicity.

In various aspects, a treatment for multiple myeloma (MM) is disclosed that incorporates several concepts. Nanoparticle delivery is incorporated to enhance efficacy and to reduce toxicity. In addition, tumor-associated endothelium is targeted for specific delivery of the therapeutic compounds to the tumor area, rather than specifically targeting the tumor cells directly. Further, the delivery of a chemotherapy compound including, but not limited to, bortezomib (BTZ) is synchronized with the delivery of a BMME-disrupting agent including, but not limited, to a ROCK inhibitor to overcome the BMME-induced drug resistance. In various aspects, targeting the BMME with BTZ and ROCK inhibitor-loaded liposomes loaded with P-selectin glycoprotein ligand 1 (also referred to herein as PSGL-1-targeted liposomes) showed the most profound efficacy as compared to the drugs in free form, non-targeted liposomes, and single-agent control groups, and reduced the severe side effects of BTZ. These results support the basis of using liposomal BTZ formulations for the treatment of MM patients.

One of the main culprits associated with relapse in MM patients is the BMME, which induces pleiotropic signaling that confers tumorigenesis and drug resistance to MM cells by direct interaction. BTZ is the first FDA-approved PI and one of the frontline regimens used for the treatment of MM patients. Despite the demonstrated clinical success of BTZ, dose-limiting toxicities and the development of drug resistance hinder the ability of BTZ to eradicate MM.

Without being limited to any particular theory, better efficacy and reduced toxicity of treatment are achieved by encapsulating the chemotherapy in a nanoparticle and by adding targeting elements that increase the specific accumulation of the particles (and chemotherapy payloads) to the tumor. BTZ loaded into a chitosan nanoparticle and decorated with anti-CD38 antibodies improves the specific accumulation of BTZ in MM cells, which overexpress CD38, and reduces the toxic side effects of BTZ in normal tissue. However, the first barrier nanoparticles face in the tissue are endothelial cells in the blood vessels adjacent to the tumor rather than the tumor cells themselves. In various aspects, the tumor-associated endothelium is targeted in the tumor area.

PSGL-1 (the natural ligand of P-selectin) plays a critical role in the interaction of MM cells with endothelial cells and is involved in adhesion and homing of MM cells to the bone marrow (BM). Without being limited to any particular theory, it is thought that the receptor of PSGL-1 (P-selectin) is highly and specifically expressed on the endothelium in the vicinity of MM cells. Consequently, P-selectin is used as a unique target to guide specific drug delivery to the tumor areas accompanying MM.

In addition to limitations of cancer therapies imposed by lack of specificity as described above, the development of drug resistance over time imposed an additional limitation. Without being limited to any particular theory, the interaction between MM cells and BMME plays a crucial role in the development of resistance to therapy. Administering a BMME-disrupting agent, such as the CXCR4 inhibitor AMD3100, re-sensitizes MM to BTZ in vivo. The combination treatment of AMD3100 (also referred to herein as Plerixafor) and BTZ was evaluated in a clinical trial with an encouraging 510% overall response rate in relapsed MM patients.

However, the administration of Plerixafor concurrently with the independent administration of BTZ may face at least several challenges. The pharmacokinetic (PK) half-life of Plerixafor is between 3-5 hours, which severely hinders efficient drug administration because the drug needed to be infused for six consecutive days, which causes discomfort to patients. In addition, the PK half-life of Plerixafor is significantly shorter than the PK half-life of BTZ (40 hours), making it difficult to determine an effective combinatorial and synchronized treatment schedule. Additionally, the combination treatment of Plerixafor and BTZ induces various adverse side effects. Without being limited to any particular theory, a nanoparticulate delivery system with dual loading of chemotherapy and BMME-disrupting agents will overcome the PK problem and ensure the simultaneous delivery of the two agents to the desired target.

In one aspect, a composition for the treatment of MM is disclosed that includes liposomes loaded with a chemotherapy compound and a BMME-disrupting agent. Without being limited to any particular theory, it is thought that the chemotherapy compound and the BMME-disrupting agent should be relatively matched with respect to the sites of action and release kinetics to enhance the effectiveness of the combined compounds. However, the delivery of the chemotherapy compound and the BMME-disrupting agent using the same liposomal vehicle may ameliorate at least some of the shortcomings of treatment efficacy associated with differing PK characteristics that arise with separate administration of the chemotherapy compound and the BMME-disrupting agent. “Site of action”, as used herein, refer to a particular region contacted or accessed by a compound to exert a biological effect. “Release kinetics”, as used herein, refer to any one or more pharmacokinetic (PK) characteristics of a compound, such as PK half-life. In one aspect, both the chemotherapy and the BMME-disrupting agent act on extracellular targets, such as extracellular receptor domains. In another aspect, both the chemotherapy and the BMME-disrupting agent act on intracellular targets such as kinases or other enzymes or subcellular structures within a cell.

In one aspect, the composition for the treatment of MM includes liposomes loaded with the chemotherapy compound BTZ as well as the BMME-disrupting agent in the form of a ROCK inhibitor. Without being limited to any particular theory, Plerixafor was not included in this composition because Plerixafor is a CXCR4 inhibitor released into the extracellular milieu to inhibit the extracellular domain of CXCR4, whereas BTZ is internalized into the cell to inhibit the proteasome. By contrast, the ROCK inhibitor used as the BMME-disrupting agent in this composition acts on a kinase inside the cell to inhibit the interaction between MM cells and their BMME, with a similar overall effect as Plerixafor.

In another aspect, the loaded liposomes of the treatment the composition described above are decorated or functionalized with P-selectin glycoprotein ligand 1 (PSGL-1). Without being limited to any particular theory, P-selectin is overexpressed in MM-associated endothelium, and PSGL-1-targeted liposomes preferentially bind to the MM-associated endothelium. As described in the Examples below, PSGL-1-targeted delivery of liposomal TME-disrupting agent and bortezomib showed higher efficacy and lower toxicity compared to corresponding free (non-targeted, non-liposomal) drug compositions. Disrupting the interaction between MM cells and the BMME using targeted nanoparticles or liposomes is thought to improve efficacy and to reduce side effects associated with BTZ.

In one aspect, as described in the Examples below, PSGL-1-targeted liposomes loaded with BTZ and Y27632, which incorporate the concepts of targeting to MM-associated endothelium and coordinating the delivery of chemotherapy compounds and BMME-disrupting agents, demonstrate better specificity, enhanced efficacy, and reduced side effects relative to non-targeted and/or non-liposomal administration of the same compounds. These results support the basis of using liposomal BTZ formulations for the treatment of MM patients.

Additional descriptions of the elements of the targeted liposomal MM treatment composition are provided below.

### I. Liposomes

Liposomes, as used herein, refer to spherical vesicles made of a lipid bilayer including, but not limited to, a phospholipid bilayer, that is capable of encapsulating hydrophilic compounds in an aqueous core or hydrophobic compounds within a lipid bilayer. Drugs loaded within liposomes can provide prolonged systemic circulation time, decreased drug toxicity, and enhanced drug delivery efficacy.

In various aspects, liposomes of the disclosed composition for the treatment of MM may be composed primarily of vesicle-forming lipids. Vesicle-forming lipids form spontaneously into bilayer vesicles in water. Non-limiting examples of vesicle-forming lipids include phospholipids that form a vesicle with a hydrophobic moiety of each phospholipid in contact with the interior of the lipid bilayer, a hydrophobic region of the bilayer membrane, and a phospho head group moiety oriented toward the exterior, polar surface region of the bilayer membrane forming the vesicle as well as toward the interior, polar surface region enclosing the aqueous core of the vesicle. Lipids capable of stable incorporation into lipid bilayers, such as cholesterol and its various analogs, can also be used in the liposomes in some aspects. In various aspects, the vesicle-forming lipids are preferably lipids having two hydrocarbon chains, including but not limited to acyl chains, and a head group that may be either polar or nonpolar. Non-limiting examples of synthetic vesicle-forming lipids and naturally-occurring vesicle-forming lipids include phospholipids, such as phosphatidylcholine, phosphatidylethanolamine, phosphatidic acid, phosphatidylinositol, and sphingomyelin, where the two hydrocarbon chains are typically between about 14-22 carbon atoms in length and have varying degrees of unsaturation. Other non-limiting examples of suitable vesicle-forming lipids include glycolipids, cerebrosides, and sterols, such as cholesterol.

In various aspects, the vesicle-forming lipids may be selected to achieve a specified degree of fluidity or rigidity, to control the stability of the liposome in serum, to control the rate of release of the entrapped agent in the liposome, and any other suitable liposome characteristic. In one aspect, liposomes having a more rigid lipid bilayer, or a gel-phase bilayer, may be achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures. In another aspect, lipid fluidity may be achieved by the incorporation of a relatively fluid lipid, typically one having a lipid phase with a relatively low gel to liquid-crystalline phase transition temperature, e.g., at or below room temperature.

In various other aspects, the lipid bilayer of the liposomes may include one or more vesicle-forming lipids covalently linked to hydrophilic polymers. By way of non-limiting example, vesicle-forming lipids covalently linked to hydrophilic polymers are described in U.S. Pat. No. 5,013,556. Without being limited to any particular theory, polymer-derivatized lipids within the lipid bilayer of a liposome may form a surface coating of hydrophilic polymer chains around the liposome. This surface coating of hydrophilic polymer chains may enhance the in vivo blood circulation lifetime of the liposomes when compared to liposomes lacking such a coating.

Non-limiting examples of polymer-derivatized lipids include mPEG-phosphatidylethanolamine compounds that include methoxy(polyethylene glycol) (mPEG) at various mPEG molecular weights ranging from about 350 to about 5,000 Daltons that are covalently linked to a phosphatidylethanolamine such as dimyristoyl phosphatidylethanolamine, dipalmitoyl phosphatidylethanolamine, distearoyl phosphatidylethanolamine (DSPE), or dioleoyl phosphatidylethanolamine. Other non-limiting examples of polymer-derivatized lipids include lipopolymers of mPEG-ceramide. In other aspects, the lipid bilayer may include “neutral” lipopolymers including, but not limited to polymer-distearoyl conjugates.

In various aspects, the liposomes of the disclosed composition for the treatment of MM further incorporate additional components including, but not limited to, treatment compounds, efficacy-enhancing compounds, and targeting moieties. The additional may be incorporated into the liposomes of the MM treatment composition using any suitable method known in the art without limitation. In various aspects, the method of incorporation may be selected based on one or more characteristics of the additional component including, but not limited to, the hydrophobicity and/or polarity of the additional component, and the nature of the chemical interaction of the additional component with the tumor cell and/or surrounding environment. By way of non-limiting example, additional components that are water-soluble/hydrophilic or polar compounds may be encapsulated within an aqueous center of the liposomes. In other aspects, hydrophobic or non-polar compounds may be encapsulated within the non-polar inner region of the lipid bilayer membrane of the liposome. By way of non-limiting example, a compound may be conjugated to PEG or a lipid, such as a phospholipid, for incorporation into the liposome lipid bilayer.

In additional aspects, additional components that interact directly with exposed elements of tumor cells, such a targeting moiety as described below, may be attached or coupled to the outer surface of the liposome. By way of non-limiting example, a targeting moiety may be coupled to the outer surface of a liposome by including the targeting moiety in a lipopolymer modified to form a lipid-polymer-targeting moiety conjugate that is incorporated into the lipid bilayer of the liposome.

In some aspects, the MM treatment formulation that includes the liposomes loaded with a treatment compound, efficacy enhancing compound, and targeting moieties may be lyophilized. The liposomal formulations may be configured to maintain stability during lyophilization, and once lyophilized, may remain stable when stored at room temperature for periods of up to six months or more. In other aspects, the MM treatment formulation may further include a lyoprotectant, including, but not limited to, sucrose or trehalose.

Lyophilized formulations can be readily reconstituted prior to administration by adding an aqueous solvent. The reconstitution solvent can be suitable for pharmaceutical administration (e.g., for parenteral administration to a subject) Examples of suitable reconstitution solvents include, without limitation, water, saline, and phosphate-buffered saline (PBS).

Liposomal formulations including the compounds described herein can be formed using any suitable method for preparing and/or loading liposomes. By way of non-limiting example, a treatment compound and/or the efficacy-enhancing compound described below and one or more vesicle-forming lipids can be dissolved in a suitable solvent, and the solvent can be evaporated to form a lipid film. The lipid film can be hydrated with an aqueous solution (e.g., having a pH of from 7-9) to form liposomes comprising the entrapped compound.

After liposome formation, the liposomes can be sized to obtain a population of liposomes having a substantially homogeneous size range, for example from about 10 nm to about 500 microns. In other aspects, the population of liposomes may have sizes ranging from 10 nm to 30 nm, 20 nm to 40 nm, 30 nm to 50 nm, 40 nm to 60 nm, from 50 nm to 70 nm, from 60 nm to 80 nm, from 70 nm to 90 nm, from 80 nm to 100 nm, from 90 nm to 110 nm, from 100 nm to 120 nm, from 110 nm to 130 nm, from 120 nm to 140 nm, from 130 nm to 150 nm, from 140 nm to 160 nm, from 150 nm to 170 nm, from 160 nm to 180 nm, from 170 nm to 190 nm, from 180 nm to 200 nm, from 190 nm to 210 nm, from 200 nm to 220 nm, from 210 nm to 230 nm, from 220 nm to 240 nm, from 230 nm to 250 nm, from 145 nm to 155 nm, from 140 nm to 160 nm, or from 125 nm to 175 nm. Liposomes can be sized by any suitable method, such as by extrusion through a series of membranes having a selected uniform pore size (e.g., polycarbonate membranes having a selected uniform pore size in the range of 0.03 to 0.2 micron). The pore size of the membrane corresponds roughly to the largest sizes of liposomes produced by extrusion through that membrane, particularly where the preparation is extruded two or more times through the same membrane. Homogenization methods can also be used to prepare liposomes having sizes of 100 nm or less.

In various aspects, the size distribution of the liposomal composition may be assessed using any known quantity including, but not limited to, polydispersity index (PDI). Typically, PDI values range from 0.05, corresponding to extremely highly monodisperse size distributions, to 0.7, corresponding to a broad size distribution. In various aspects, the liposomal compositions may be characterized as having a PDI of 0.3 or less.

In some embodiments, the liposomes in the formulation can have an average particle size, as measured by dynamic light scattering, ranging from 50 nm to 250 nm (e.g., from 50 nm to 200 nm, from 75 nm to 150 nm, from 90 nm to 150 nm, from 120 nm to 150 nm, from 100 nm to 130 nm, from 90 nm to 110 nm, from 100 nm to 120 nm, from 110 nm to 130 nm, from 120 nm to 140 nm, from 130 nm to 150 nm, from 140 nm to 160 nm, from 150 nm to 170 nm, from 160 nm to 180 nm, from 170 nm to 190 nm, from 180 nm to 200 nm, from 190 nm to 210 nm, from 200 nm to 220 nm, from 210 nm to 230 nm, from 220 nm to 240 nm, or from 230 nm to 250 nm). In some embodiments, the liposomes in the formulation can have a zeta potential ranging from −50 mV to 0 mV (e.g., from −50 mV to −40 mV, from −45 mV to −35 mV, or from −40 mV to −30 mV, from −35 mV to −25 mV, from −30 mV to −20 mV, from −25 mV to −15 mV, from −20 mV to −10 mV, from −15 mV to −5 mV, or from −10 mV to 0 mV).

After sizing, unencapsulated compounds can be removed by a suitable technique, such as dialysis, centrifugation, tangential-flow diafiltration, size exclusion chromatography, or ion exchange to achieve a suspension of liposomes having a high concentration of entrapped compounds in the liposomes and little to no compound in solution outside of the liposomes. Also after liposome formation, the external phase of the liposomes can be adjusted, if desired, by titration, dialysis, or the like, to an appropriate pH.

### II. Treatment Compounds

In various aspects, the disclosed composition for the treatment of MM includes a MM treatment compound as one additional component. In various aspects, the MM treatment compound includes any cytotoxic or other compound that kills or otherwise adversely affects cancer cells. Non-limiting examples of MM treatment compounds suitable for inclusion in the liposomes of the disclosed treatment composition include chemotherapy compounds, immunomodulating agents, proteasome inhibitors, histone deacetylase (HDAC) inhibitors, and nuclear export inhibitors. Non-limiting examples of suitable chemotherapy compounds include Melphalan, Vincristine, Cyclophosphamide, Etoposide, Doxorubicin, and Bendamustine. Non-limiting examples of suitable immunomodulating agents include Thalidomide and Lenalidomide. Non-limiting examples of suitable proteasome inhibitors include Bortezomib, Carfilzomib, and Ixazomib. Non-limiting examples of suitable histone deacetylase (HDAC) inhibitors include Panobinostat. Non-limiting examples of suitable nuclear export inhibitors include Selinexor. In one aspect, the MM treatment compound included within the liposome of the disclosed composition is Bortezomib (BTZ).

### III. Efficacy-Enhancing Compounds

In various aspects, the disclosed composition for the treatment of MM includes an efficacy-enhancing compound as an additional component. Without being limited to any particular theory, the efficacy-enhancing compound is included to enhance the efficacy of the MM treatment compound by modifying one or more aspects of the tumor environment. In various aspects, the efficacy of the MM treatment compound may be enhanced by one or more means including, but not limited to, sensitization of tumor cells to the MM treatment compound, development of resistance to the MM treatment compound, disruption of chemical signaling between tumor cells and the surrounding cell environment, and any other aspect of the tumor environment relevant to the treatment of MM.

The efficacy-enhancing compound may be incorporated into the liposome using any means known in the art without limitation. In various aspects, the efficacy-enhancing compound may be incorporated into various parts of the liposome depending on one or more characteristics of the efficacy-enhancing compound as described above.

### IV. Targeting Moieties

In various aspects, the liposomes of the disclosed MM treatment composition further include at least one targeting moiety coupled to an exposed outer surface of each liposome. Without being limited to any particular theory, at least one targeting moiety is configured to preferentially bind, complex with, or otherwise couple to, a ligand specifically associated with the tumor cells and/or cells within the immediate vicinity of the tumor cells to enable targeted administration of the MM treatment compound and the efficacy-enhancing compound carried by the liposomes. In various aspects, the targeting moiety may be an antagonist of a target cell surface receptor. Non-limiting examples of suitable targeting moieties include VLA-4 antagonists. In some aspects, the targeting moiety is a VLA-4 antagonist peptide (“VLA-4-pep”) configured to bind to fibronectin and/or to VLA-4. Other non-limiting examples of VLA-4 antagonists suitable for use as targeting moieties include peptide sequences with a consensus LDV sequence, cyclic peptides with an RCD motif, peptides derived from fibronectin CS-1, peptides derived from fibronectin RGD tripeptide, peptides derived from fibronectin RGD or vascular cell adhesion molecule-1, peptides derived from anti-α4 monoclonal antibody, and any other suitable VLA-4 antagonist known in the art. In various other aspects, the targeting molecules may be antagonists and/or ligands of other receptors. Non-limiting examples of other suitable targeting molecules include, but are not limited to, folates configured to bind folate receptor, RGD peptide sequences against the αvβ3 integrin, peptide antagonists of the Human Epidermal Growth Factor Receptor 2 (HER2), and P-selectin glycoprotein ligand 1 (PSGL-1) configured to bind to P-selectin. Additional examples of targeting moieties include molecules having binding affinity to receptors for CD4, folate, insulin, LDL, vitamins, transferrin, asialoglycoprotein, selectins, such as E, L, and P selectins, Flk-1,2, FGF, EGF, integrins, in particular, α4β1 αvβ3, αvβ1 αvβ5, αvβ6 integrins, HER2, and others. In various other aspects, the targeting moieties may be ligands that may be proteins and peptides, including, but not limited to antibodies and antibody fragments, such as F(ab′)2, F(ab)2, Fab′, Fab, Fv (fragments consisting of the variable regions of the heavy and light chains), and scFv (recombinant single chain polypeptide molecules in which light and heavy variable regions are connected by a peptide linker), and the like. The ligand may also be a small molecule peptidomimetic. It will be appreciated that a cell surface receptor, or fragment thereof, can serve as the ligand. Other non-limiting examples of suitable targeting ligands include vitamin molecules (e.g., biotin, folate, and cyanocobalamine), oligopeptides, and oligosaccharides. In one aspect, the targeting molecule is P-selectin glycoprotein ligand 1 (PSGL-1) configured to bind to P-selectin, which is overexpressed in MM-associated endothelium as described above and in the Examples provided below.

In various other aspects, the targeting moiety may be selected based on at least one desired characteristic of the MM treatment composition. By way of non-limiting example, if the MM treatment compound and the efficacy-enhancing compound are configured to modulate processes within the interior of a tumor cell, the targeting moiety may selectively bind to a surface cell receptor known to internalize bound ligands.

The targeting moieties may be attached to the exposed external surface of the liposomes using any known method without limitation. In one aspect, lipopolymers may be prepared, in which the polymer portion may be functionalized for subsequent reaction with a selected ligand. In other aspects, functionalized polymer-lipid conjugates may also be obtained commercially, such as end-functionalized PEG-lipid conjugates. The linkage between the ligand and the polymer can be a stable covalent linkage or a releasable linkage that is cleaved in response to a stimulus, such as a change in pH or the presence of a reducing agent.

In one aspect, PSGL-1 targeting moieties may be bound to the surface of liposomes using carbodiimide chemistry. In this aspect, the liposomes are suspended in a solution of 0.25 M EDC and 0.25 M NHS (in water) and incubated for 10 minutes at room temperature, followed by incubation of PSGL-1 within the colloidal liposome suspension, as described in additional detail in the Examples below.

### V. Formulations

In various aspects, the targeted liposomal compositions described above may be used to prepare therapeutic pharmaceutical compositions, for example, by combining the liposomes with a pharmaceutically acceptable diluent, excipient, or carrier. The liposomes described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical, or subcutaneous routes.

The liposomes described herein may be systemically administered in combination with a pharmaceutically acceptable vehicle, such as an inert diluent or an assimilable edible carrier. In various aspects, the liposomes may be administered intravenously or intraperitoneally by infusion or injection. Solutions of the nanoparticles can be prepared in water, optionally mixed with a nontoxic surfactant. Dispersions can be prepared in glycerol, liquid polyethylene glycols, triacetin, or mixtures thereof, or a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising lyophilized liposomes for the extemporaneous preparation of sterile injectable or infusible solutions or dispersions. The ultimate dosage form for injection or infusion should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the nanoparticles in the required amount in the appropriate solvent or carrier with various other ingredients enumerated above, as required, optionally followed by filter sterilization. In the case of sterile powders for the preparation of sterile injectable solutions, methods of preparation can include vacuum drying and freeze-drying techniques, which yield a powder of the nanoparticles plus any additional desired ingredient present in the composition.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze-dried (lyophilized) condition requiring only the condition of the sterile liquid carrier, for example, water for injections, prior to use. Extemporaneous injection solutions and suspensions can be prepared from sterile powder, granules, tablets, etc. it should be understood that in addition to the ingredients particularly mentioned above, the compositions disclosed herein may include other agents conventional in the art having regard to the type of formulation in question.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required liposome size and/or by the use of surfactants. Optionally, the prevention of the action of microorganisms can be brought about by various other antibacterial and antifungal agents, tier example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

Doses and a dosing regimen for the compounds and formulations described herein will depend on the cancer being treated, the stage of the cancer, the size and health of the patient, and other factors readily apparent to an attending medical caregiver. By way of non-limiting example, previously published clinical studies directed to the administration of the proteasome inhibitor bortezomib (BTZ), Pyz-Phe-boroLeu (PS-341), may be used to provide guidance for selecting suitable dosages and dosing regimens. By way of non-limiting example, one previously published study determined that the maximum tolerated dose of bortezomib given intravenously to patients with solid tumors once or twice weekly was 1.3 mg/m² (Orlowski, R. Z. et al., Breast Cancer Res. 5:1-7 (2003)). By way of another non-limiting example, another previously published study determined that bortezomib administered as an intravenous bolus on days 1, 4, 8, and 11 of a 3-week cycle had a maximum tolerated dose of about 1.56 mg/m² (Vorhees, P. M. et al., Clinical Cancer Res. 9:6316 (2003)). Without being limited to any particular theory, because liposomal BTZ is likely less toxic than free BTZ, as demonstrated in the Examples below, the clinical dose of liposomal BTZ may be several times higher than a corresponding dose of BTZ in free (non-liposomal) form.

### VI. Methods of Administration

The compounds and formulations described herein can be administered parenterally (e.g., by intravenous administration or subcutaneous administration). It will be appreciated that the formulation can include any necessary or desirable pharmaceutical excipients to facilitate delivery. The compounds and formulation disclosed herein can also be administered orally, by intraperitoneal injection, by intramuscular injection, intratumoral injection, and by airway administration as a micronized solid or liquid aerosol.

The term “administration” and variants thereof (e.g., “administering” a compound) in reference to a compound as described herein means introducing the compound or a formulation thereof into the system of a subject in need of treatment. When a compound as described herein or a formulation thereof is provided in combination with one or more other active agents (e.g., a cytotoxic agent, etc.), “administration” and its variants are each understood to include the concurrent and sequential introduction of the compound or formulation thereof and other agents.

In vivo application of the disclosed compounds, and formulations containing them can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or formulations can be a single administration, or at continuous or distinct intervals as can be readily determined by a person skilled in the art.

Compounds and formulations disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and formulations disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

Although the disclosed compositions and methods of treatment are presented herein as treatments for multiple myeloma, the disclosed compositions and methods of treatment are suitable for a variety of different cancers. In various aspects, the compounds or formulations described herein may be used for the treatment of cancer, and more particularly for the treatment of a tumor in a cancer patient. Non-limiting examples of cancer that may be treated using the compositioned and methods of treatment disclosed herein include stomach cancer, kidney cancer, bone cancer, liver cancer, brain cancer, skin cancer, oral cancer, lung cancer, pancreatic cancer, colon cancer, intestinal cancer, myeloid leukemia, melanoma, glioma, thyroid follicular cancer, bladder carcinoma, myelodysplastic syndrome, breast cancer, low-grade astrocytoma, astrocytoma, glioblastoma, medulloblastoma, renal cancer, prostate cancer, endometrial cancer, or neuroblastoma.

In one aspect, the cancer treated using the compositions and methods described herein is multiple myeloma. Multiple myeloma is a hematologic malignancy typically characterized by the accumulation of clonal plasma cells at multiple sites in the bone marrow. The majority of patients respond to initial treatment with chemotherapy and radiation. However, most patients typically eventually relapse due to the proliferation of resistant tumor cells. In one aspect, provided are methods for treating multiple myeloma in a subject that can comprise administering a liposome formulation comprising a targeted liposomal compound described herein.

### VII. Kits

In various aspects, the compositions disclosed herein may be included in kits provided to facilitate the administration of the disclosed compositions to a patient in need. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to the liposomes functionalized with targeting moieties and loaded with a treatment compound and an efficacy-enhancing compound as disclosed herein including, but not limited to, PSGL-1 functionalized liposomes loaded with BTZ and Y27632. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrates, and/or may be supplied as an electronic-readable medium or video. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples illustrate various aspects of the disclosure.

### Example 1: Expression of P-Selectin in MM-Associated Endothelial Cells in Human Samples

To assess the expression of P-selectin in human endothelial cells associated with multiple myeloma (MM), the following experiments were conducted.

Two MM cell lines, annotated as MM.1S and H929, were purchased from American Type Culture Collection (ATCC, Rockville, Md.). OPM-2 and green fluorescent protein-labeled and luciferase-transfected MM.1S (MM.1S-GFP-Luc) were obtained from a research lab.

Healthy bone marrow mononuclear cells (BMMNCs) were purchased from Allcells (Alameda, Calif.), and BMMNCs from MM patients were acquired from a research clinic. All cells were cultured at 37° C. and 5% CO2 in the NuAire water jacket incubator (Plymouth, Minn.). The MM cell lines were cultured in RPMI-1640 media (Corning, Tewksbury, Mass.) supplemented with 10% fetal bovine serum (FBS; Gibco, Life Technologies, Grand Island, N.Y.), 2 mmol/L of L-glutamine, 100 μg/mL penicillin, and 100 μg/mL streptomycin (Corning CellGro).

The BMMNCs from healthy and MM patients were washed in PBS supplemented with 2% FBS and stained with its respective isotype control or CD31 and CD62P mAbs for 1 hour. Cells were washed and analyzed by flow cytometry. The mAbs used for flow cytometry were purchased from Miltenyi Biotec (Bergisch Gladbach, Germany) unless otherwise noted. Endothelial cells (ECs) were gated as CD31+ cells, and the expression of P-selectin (CD62P) was quantified as the ratio of the mean fluorescence intensity (MFI) of CD62P divided by the isotype control; otherwise known as the relative MFI (RMFI). In vitro data were expressed as means±standard deviation. Results were analyzed using a student t-test or ANOVA for statistical significance and were considered significantly different for all p-values below 0.05.

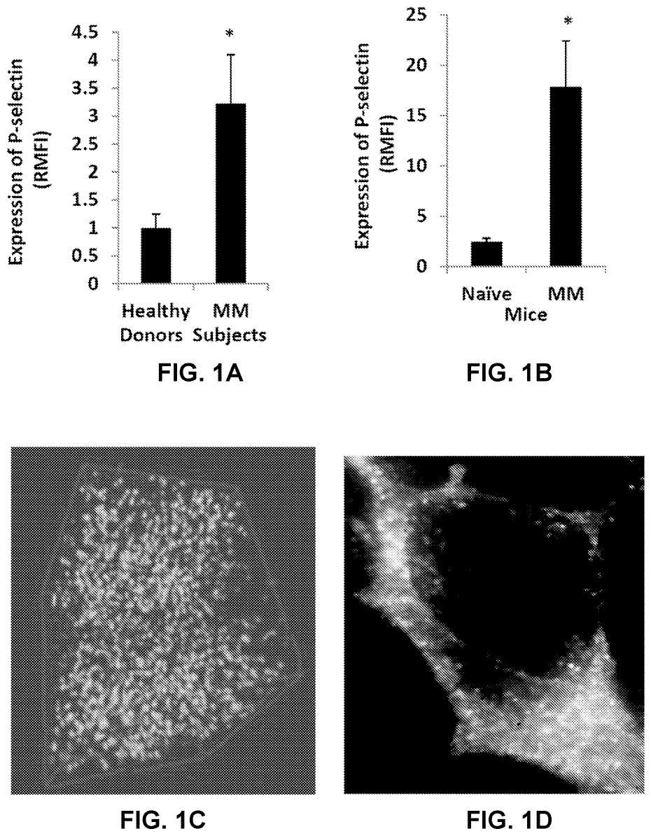

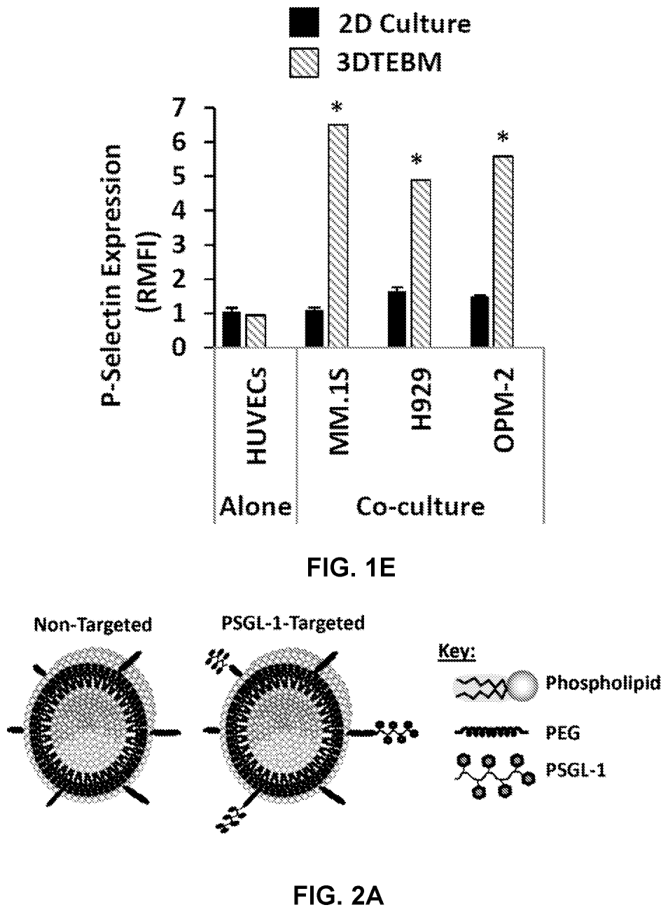

The measured expression of P-selectin was 3-fold higher on the endothelial cells (ECs) from the bone marrow (BM) of MM patients compared to P-selectin expression from ECs of healthy donors (FIG. 1A).

The results of these experiments found that P-selectin expression was highly upregulated on MM-associated endothelium in MM patients compared to healthy endothelium (see FIG. 1A).

### Example 2: Expression of P-Selectin in ECs In Vivo

To assess the expression of P-selectin in endothelial cells of mice in vivo, the following experiments were conducted.

The mice used were NCG male 50-56 day-old mice (Charles River, Wilmington, Mass.) unless otherwise stated. MM.1S-GFP-Luc cells (2×10⁶ cells/mouse) similar to those described in Example 1, were injected intravenously into five mice and tumor progression was confirmed using bioluminescent imaging (BLI) at 4 weeks post-injection as described above in Example 1. Five mice were used as control. Mice were sacrificed and femurs were flushed with PBS for the collection of bone marrow mononuclear cells (BMMNCs). P-selectin expression of the collected mouse BMMNCs was assessed using flow cytometry as described above in Example 1. The in vivo P-selectin expression data were expressed as means±standard deviation. Results were analyzed using a student t-test or ANOVA for statistical significance and were considered significantly different for all p-values below 0.05.

The measured expression of P-selectin was 7-fold higher on ECs from the BM of MM-bearing mice compared to naïve mice (FIG. 1B).

The results of these experiments found that P-selectin expression was highly upregulated on MM-associated endothelium compared to healthy endothelium in MM patients in the in vivo mouse model.

### Example 3: Expression of P-Selectin in ECs in Cell Lines In Vitro

To evaluate the effect of MM cells on P-selectin expression of endothelial cells in vitro, the following experiments were conducted.

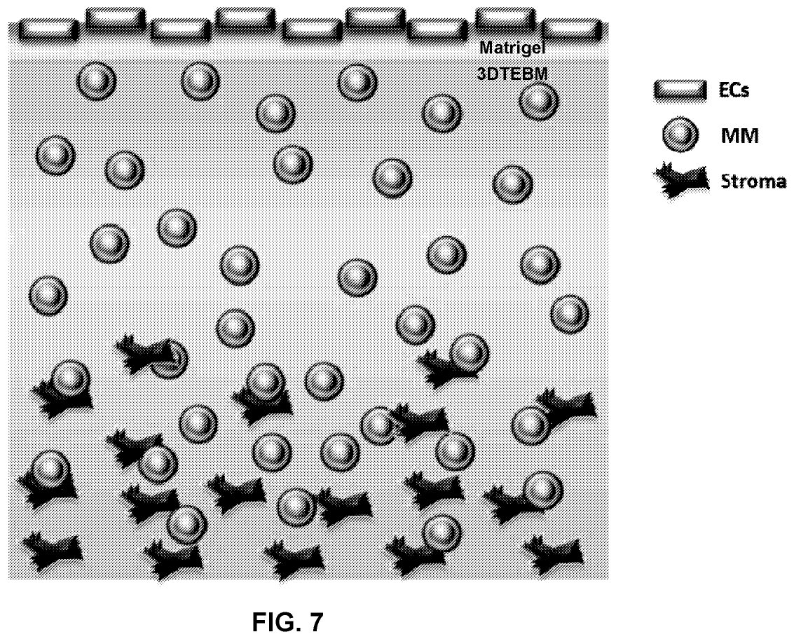

The expression of P-selectin in vitro was evaluated in HUVECs similar to those described in Example 1 using 2D and 3D tissue culture models. The HUVECs were purchased from Angio-Proteomie (Boston, Mass.) and cultured in Endothelial Growth Medium (EGM, Angio-Proteomie, Boston, Mass.) supplemented with endothelial growth supplements (including 10% FBS, recombinant growth factors, and 1% penicillin and streptomycin). To better simulate the BM niche, the 3D tissue-engineered bone marrow (3DTEBM) model was developed using BM plasma derived from MM patients. FIG. 7 depicts the organization of the cells in the 3DTEBM model. As illustrated in FIG. 7, MM and stromal cells were cultured inside the 3DTEBM while the ECs were incubated on top with matrigel.

The 2D tissue culture model included cell cultures within a 96-well plate. In each well, 1×10⁴ HUVECs pre-labeled with DiO and 1×10⁴ MSP-1 stromal cells were co-cultured with or without 3×10⁴ MM cells from one of the three MM cell lines described in Example 1: MM.1S, H929, or OPM-2.

In the 3D tissue-engineered bone marrow (3DTEBM) model, 1×10⁴ MSP-1 stromal cells with or without 3×10⁴ cells from one of three MM cell lines (MM.1S, H929, or OPM-2) were suspended in BM plasma and set to solidify into a 3D scaffold in a 96-well plate. After two hours, Matrigel (Corning, Tewksbury, Mass.) was added on top of the scaffold, and 1×10⁴ HUVECs (pre-labeled with DiO) were added on top of the Matrigel with non-supplemented EGM media.

The HUVECs and stromal cells, with and without MM cells, were cultured in the 2D and 3DTEBM tissue culture models for 24 hours. The cultures were then digested with collagenase and the cells were retrieved for flow cytometry and analysis as described in Example 1. In vitro data were expressed as means±standard deviation. Results were analyzed using a student t-test or ANOVA for statistical significance and were considered significantly different for all p-values below 0.05.

As summarized in FIG. 1E, P-selectin expression increased 6-fold for the HUVECs cultured with MM relative to HUVEC cells cultured without MM in the 3DTEBM model. The expression of P-selectin in traditional 2D cultures did not significantly increase when co-cultured with MM cell lines.

### Example 4: Confocal Imaging of the 3DTEBM Cultures of HUVECs

To obtain images of the HUVECs within the 3DTEBM tissue culture model described in Example 3, the following experiments were conducted.

1×10⁴ MSP-1 stromal cells pre-labeled with DiD and 3×10⁴ MM.1S cells pre-labeled with DiO were suspended in BM plasma to form a 3D scaffold in a Nunc Lab-Tek II Chamber Slide System (Thermofisher, Waltham, Mass.). After two hours, Matrigel was added on top of the scaffold and 1×10⁴ HUVECs pre-labeled with calcein Violet were subsequently added on top of the Matrigel. Calcein violet and lipophilic tracers (DiO and DiD) were purchased from Invitrogen (Eugene, Oreg.).

The HUVECs, stromal cells, and MM cells were cultured in the 3DTEBM for 24 hours. The samples were then imaged using an FV1000 confocal microscope with an XLUMPLFLN 20×W/1.0 immersion objective lens (Olympus, Central Valley, Pa.) with the following excitation/emission wavelengths: 405/450 nm±20 nm (calcein violet), 488/520 nm±20 nm (DiO), and 633/650±20 nm (DiD) nm.

FIG. 1C is an image of the stained cells within the 3DTEBM tissue culture model. HUVECs (cyan) were plated on top of the 3DTEBM; the stromal (red) and MM (green) cells were plated inside the 3DTEBM matrix. The MM cells were dispersed throughout the scaffold whereas the stromal cells coalesced towards the bottom of the culture, biomimicking the BM niche. In addition, the HUVECs formed a tube-like structure on top of the 3DTEBM, as illustrated in FIG. 1D.

### Example 5: Preparation and Characterization of Liposomes

To demonstrate the formation of PSGL-1-targeted liposomes, the following experiments were conducted.

The phospholipids, 1,2-dipalmitoyl-sn-glycero-3-phosphocholine (DPPC), 1,2-distearoyl-sn-gly cero-3-phosphoethanolamine-N-[methoxy(polyethylene glycol)-2000] (DSPE-mPEG2000), 1,2-distearoyl-sn-glycero-3-phosphoethanolamine-N-[succinyl(polyethylene glycol)-2000] (DSPE-PEG(2000)-succinyl) were purchased from Avanti Polar Lipids, Inc. (Alabaster, Ala.). Cholesterol (Chol), N-(3-Dimethylaminopropyl)-N′-ethylcarbodiimide hydrochloride (EDC), and N-Hydroxysuccinimide (NHS) were purchased from Sigma-Aldrich (St. Louis, Mo., USA). PSGL-1 recombinant protein was purchased from Novoprotein (Summit, N.J.).

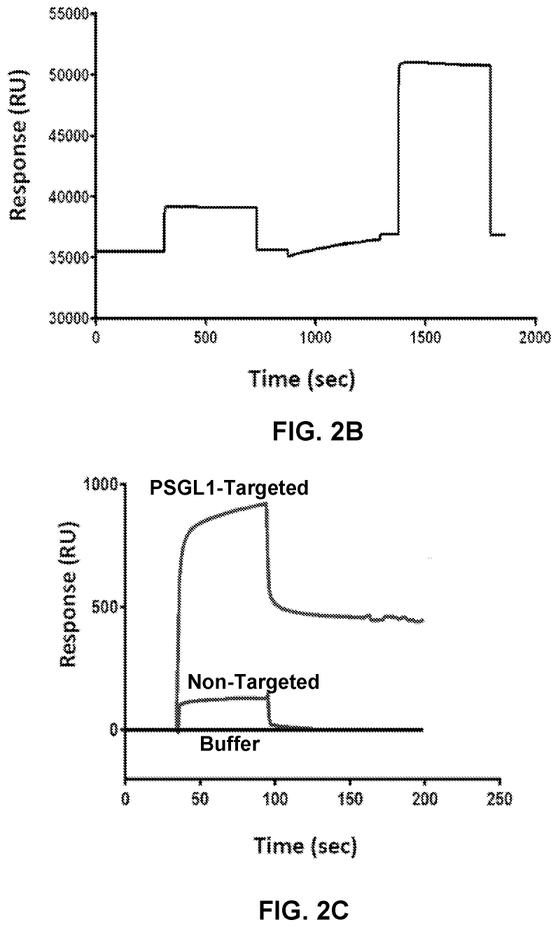

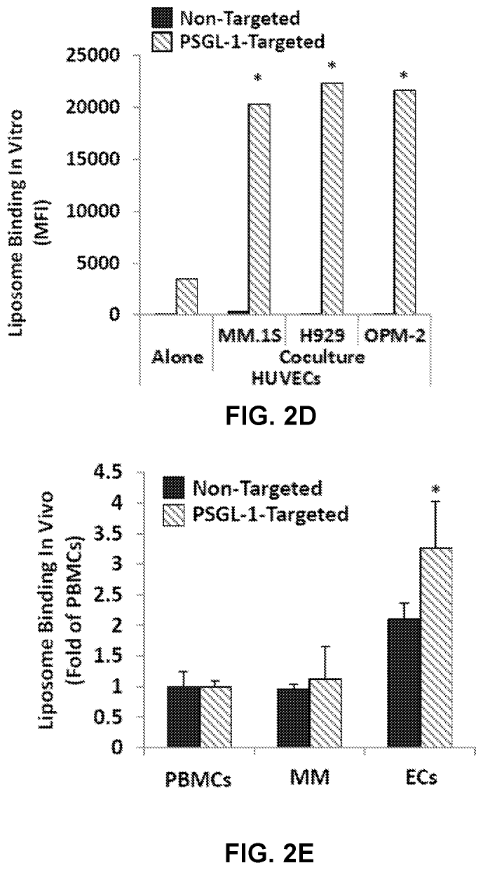

The liposomes were prepared using the thin layer evaporation method. Briefly, lipids (DPPC, Chol, DSPE-mPEG2000, and DSPE-PEG(2000)-succinyl at a molar ratio of 6:3:0.5:0.5) were dissolved in a chloroform/methanol mixture (3:1, v/v) and the solvent was then evaporated through a rotary evaporator (Heidolph, Schwabach, Germany) to form a thin lipid film. The film was then hydrated with PBS and extruded with an extruder set (Avanti Polar Lipids). Fluorescent liposomes were prepared by dissolving DiD in the organic solvent with the lipids (before film formation). A schematic diagram of the resulting non-targeted liposome is shown in FIG. 2A (left).

The conjugation of PSGL-1 to the surface of liposomes was performed using carbodiimide chemistry. Briefly, the liposomes were suspended in a solution of 0.25 M EDC and 0.25 M NHS (in water) and incubated for 10 minutes at room temperature. Then, PSGL-1 was added to the mixture and the colloidal suspension was incubated at 4° C. overnight in a light-protected environment with gentle stirring. The unbound protein was separated using Amicon Ultra Centrifugal Filter Units (100 kDa MWCO). The mean sizes, polydispersity index (PDI), and zeta-potential (ZP) were analyzed by dynamic light scattering (DLS) analysis using a Malvern Zetasizer Nano ZS (Malvern, Herrenberg, Germany).

Parameters characterizing the non-targeted and PSGL-1-targeted liposomes produced as described above are summarized in TABLE 1 below:

TABLE 1
Liposome Parameters

| | Mean Size | Polydispersity | Zeta Potential |
|---|---|---|---|
| Formulation | (nm) | Index | (mV) |
| Non-targeted | 148.4 ± 1.248 | 0.070 ± 0.015 | −41.9 ± 0.192 |
| liposomes | | | |
| PSGL-1-targeted | 146.9 ± 0.885 | 0.081 ± 0.021 | −36.1 ± 0.781 |
| liposomes | | | |

Mean ± standard deviation.

### Example 6: Affinity of PSGL-1-Targeted Liposomes to P-Selectin In Vitro

To evaluate the affinity of the PSGL-1-targeted liposomes described in Example 5 to P-selectin in vitro, the following experiments were conducted.

The affinity of PSGL-1-targeted liposomes to P-selectin was measured using a biosensor-based surface plasmon resonance (SPR) technique implemented on an automatic apparatus BIAcore T200 (GE Healthcare). Recombinant P-selectin protein was immobilized on a CM4 sensor chip surface (ligand) of the BIAcore T200 device using carbodiimide chemistry, and PSGL-1-targeted and non-targeted liposomes served as the analytes.

The immobilization of P-selectin on the CM4 sensor chip surface is shown in FIG. 2B. The data were expressed as means±standard deviation. Results were analyzed using a student t-test or ANOVA for statistical significance and were considered significantly different for all p-values below 0.05. The CM4 sensor chip with immobilized recombinant P-selectin was then contacted with buffer (control), non-targeted liposomes, and PSGL-1-targeted liposomes, both described in Example 5.

As summarized in FIG. 2C, the PSGL-1-targeted liposomes showed an 8-fold increase in binding to recombinant P-selectin compared to the non-targeted liposomes.

The results of these experiments found that the PSGL-1-targeted liposomes showed specific binding to P-selectin protein in vivo.

### Example 7: Binding of PSGL-1-Targeted Liposomes to ECs In Vitro

To assess the in vitro binding of the PSGL-1- and non-targeted liposomes, described in Example 5, to naïve and tumor-associated endothelial cells, the following experiments were conducted.

The 3DTEBM tissue culture model was used for these experiments. HUVECs pre-labeled with DiO were grown on top of the 3DTEBM tissue culture model as described in Example 3. DiD-labeled non-targeted or PSGL-1-targeted liposomes were cultured with the HUVECs for 2 hours. The 3D cultures were then digested, washed, and analyzed via flow cytometry as described in Example 3. The in vitro data were expressed as means±standard deviation. Results were analyzed using a student t-test or ANOVA for statistical significance and were considered significantly different for all p-values below 0.05.

As summarized in FIG. 2D, the non-targeted liposomes had negligible binding to the endothelium, and the PSGL-1-targeted liposomes had significantly higher binding (7-fold) to the tumor-associated endothelium compared to the naïve endothelium. These results are in agreement with other previous findings showing the overexpression of P-selectin in tumor-associated endothelium in glioblastoma, lung, ovarian, lymphoma, breast, and other cancer subtypes, which suggests that targeting with PSGL-1 can be used as a general platform for targeting the tumor-associated endothelium in other cancer subtypes.

### Example 8: Binding of PSGL-1-Targeted Liposomes to ECs In Vivo

To assess the binding of the PSGL-1- and non-targeted liposomes described in Example 5 to naïve and tumor-associated endothelial cells in vivo, the following experiments were conducted.

The mouse model described in Example 2 was used for these experiments. MM.1S-GFP-Luc cells (2×10⁶ cells/mouse) were injected intravenously into ten mice and tumor progression was confirmed using BLI at 4 weeks post-injection. Mice were then injected intravenously with DiD-labeled non-targeted liposomes or PSGL-1-targeted liposomes (2 mg/mL of lipids; 5 mice per group). Mice were sacrificed and femurs were flushed with PBS for the collection of BMMNCs. In addition, a blood sample was collected from the tail vein of each mouse, lysed with 1× red blood cell lysis buffer (BioLegend, San Diego, Calif.) using the manufacturer's instructions to isolate the peripheral blood mononuclear cells (PBMCs) of each mouse. The isolated PBMCs were cultured with non-targeted and PSGL-1-targeted liposomes as a control.

The binding of the non-targeted and PSGL-1-targeted liposomes to the BMMNCs and PBMCs of each mouse were analyzed via flow cytometry. The in vitro data were expressed as means±standard deviation. Results were analyzed using a student t-test or ANOVA for statistical significance and were considered significantly different for all p-values below 0.05.

As summarized in FIG. 2E, the PSGL-1-targeted liposomes had higher binding to the MM-associated endothelium, compared to the non-targeted liposomes.

### Example 9: HPLC Detection of BTZ and Y27632

To develop a means of detecting BTZ and Y27632, the following experiments were conducted.

BTZ and Y27632 were purchased from MedKoo Biosciences (Morrisville, N.C.). The BTZ and Y27632 were analyzed using high-performance liquid chromatography (HPLC, Agilent 1100 series, Santa Clara, Calif.) with a reverse phase C-18 column (Agilent Zorbax Eclipse XDB-C18, 4.6 mm×150 mm).

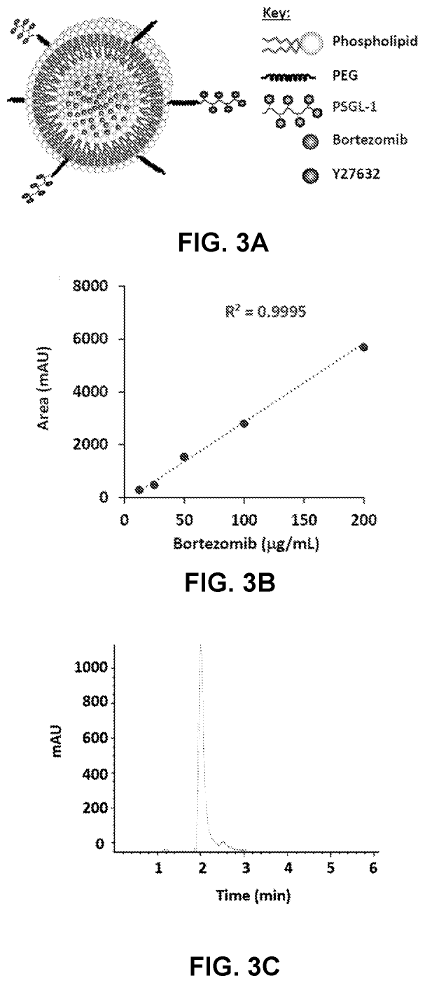

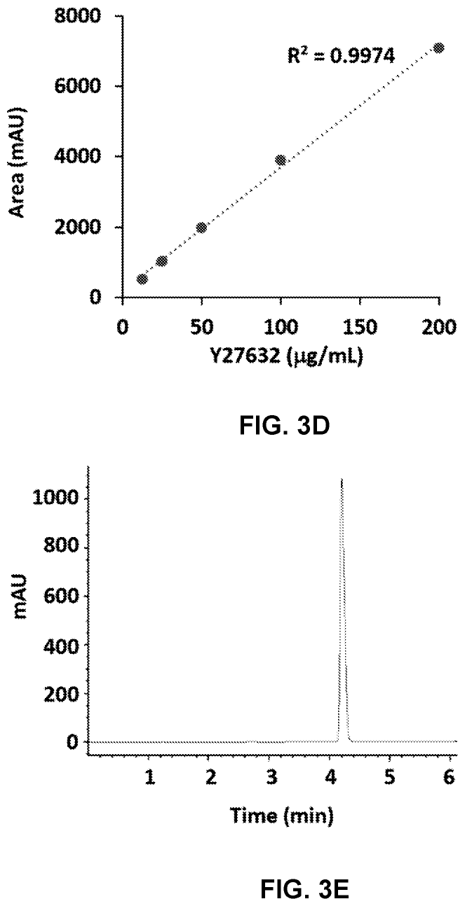

For the detection of BTZ, a 50% acetonitrile solution in water containing 0.1% trifluoroacetic acid (TFA) was used as the mobile phase at a flow rate of 1 mL/min. A calibration curve was obtained by plotting the area under the curve (AUC) of the BTZ HPLC peak (at retention time=2.2 min, λ=260 nm) for a concentration range of 0 to 200 μg/mL, shown illustrated in FIG. 3B.

For the detection of Y27632, a gradient of acetonitrile/water containing 0.1% TFA was used as the mobile phase at a flow rate of 1 mL/min. The percentile of acetonitrile in the mobile phase was 0% (at 0-3 min), then increased gradually to 33% water (3 to 3.5 min), and decreased gradually back to 0% (3.5 to 7 min). A calibration curve was obtained by plotting the area under curve (AUC) of the Y27632 HPLC peak (at retention time=4 min, λ=260 nm) for a concentration range of 0 to 200 μg/mL, shown illustrated in FIG. 3D.

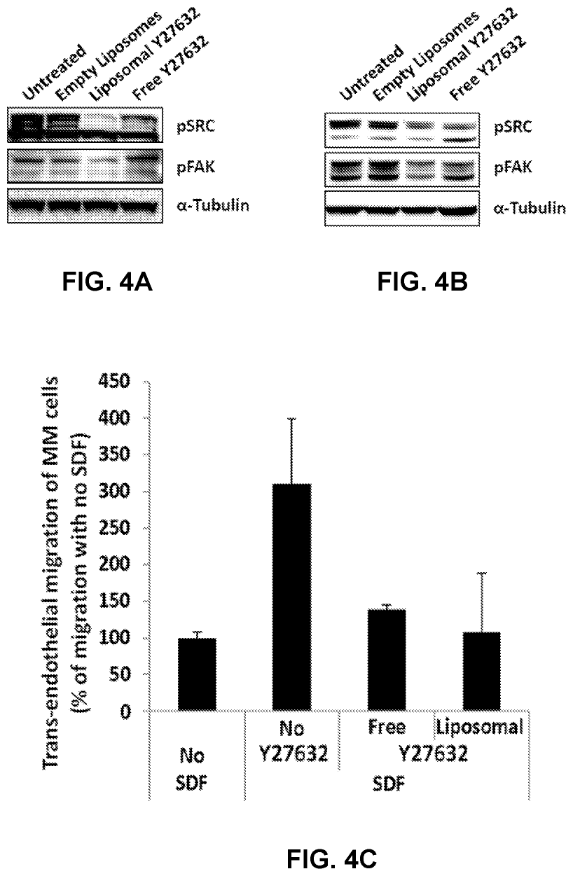

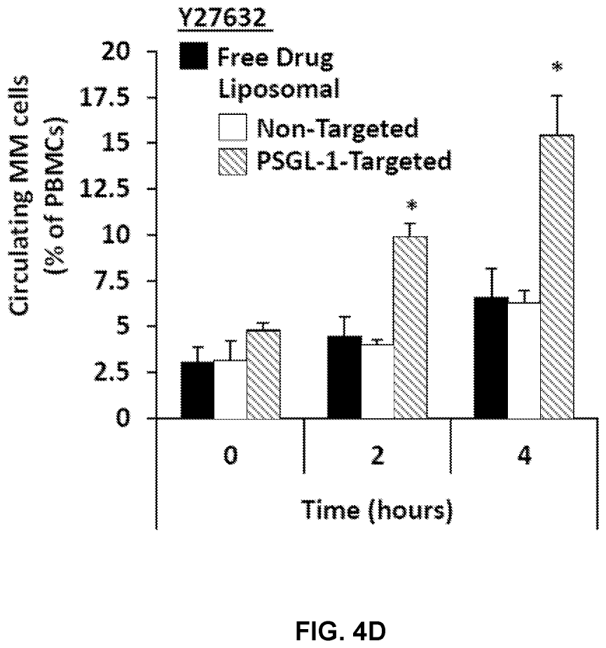

The retention times of BTZ and Y27632 were determined to be 2.1 min (FIG. 3C) and 4.1 min (FIG. 3E), respectively, with linear calibration curves in the range of 12.5-200 μg/ml and linear correlation coefficients of 0.99.

### Example 10: Drug Loading in Liposomes and Evaluation of Drug Entrapment Efficiency

To evaluate the loading of a therapeutic compound and a BMME-disrupting agent into the targeted liposomes described in Example 5, the following experiments were conducted.

The drug-loaded liposomes were prepared by incorporating BTZ (a chemotherapy compound) and/or Y27632 (a ROCK inhibitor compound) into the liposome synthesis process described in Example 5. In these experiments, BTZ was incorporated into the lipid bilayer and Y27632 was incorporated into the hydrophilic core of the liposomes. The BTZ was incorporated into the lipid bilayer by adding the BTZ to the lipid mixture in the organic solvent (before film formation), and Y27632 was incorporated into the hydrophilic core of the liposome by dissolving the Y27632 into the PBS used for hydration after film formation.

One example of a drug-loaded liposome is illustrated schematically in FIG. 3A. The physical characterization of the loaded liposomes is summarized in Table 2 below.

TABLE 2
Parameters for Drug-Loaded Liposomes.

| | Mean Size | Polydispersity | Zeta Potential |
|---|---|---|---|
| Formulation | (nm) | Index | (mV) |
| Non-targeted | 146.3 ± 1.914 | 0.153 ± 0.010 | −44.4 ± 0.503 |
| empty liposomes | | | |
| Non-targeted BTZ | 168.2 ± 1.007 | 0.109 ± 0.006 | −45.9 ± 1.29 |
| liposomes | | | |
| Non-targeted | 138.3 ± 1.027 | 0.051 ± 0.009 | −32.1 ± 0.061 |
| Y27632 liposomes | | | |
| Non-targeted | 163.2 ± 2.372 | 0.126 ± 0.017 | −41.4 ± 1.42 |
| multi-drug | | | |
| liposomes | | | |
| PSGL-1-targeted | 147.7 ± 0.9960 | 0.157 ± 0.017 | −33.9 ± 1.27 |
| empty liposomes | | | |
| PSGL-1-targeted | 172.0 ± 2.826 | 0.101 ± 0.037 | −35.4 ± 2.86 |
| BTZ liposomes | | | |
| PSGL-1-targeted | 172.0 ± 2.826 | 0.101 ± 0.037 | −35.4 ± 2.86 |
| Y27632 liposomes | | | |
| PSGL-1 targeted | 172.6 ± 4.883 | 0.129 ± 0.027 | −29.4 ± 0.883 |
| multi-drug | | | |
| liposomes | | | |

Mean ± standard deviation.

To evaluate loading efficiency, the liposomes were centrifuged at 38,000 rpm at 4° C. for 1 hour using a Beckman Optima™ XPN ultracentrifuge equipped with an SW 50.1 fixed angle rotor (Beckman Coulter Inc., Fullerton, Calif., USA). The amount of BTZ and Y27632 in the supernatant was evaluated by HPLC as described in Example 9. The entrapment efficiency (EE) was calculated according to the following equation:

EE=D_{T}−D_{U}/D_{T}×100

where D_{T} represents the total amount of drug added to the formulation during the preparation, and D_{U} represents the amount of unincorporated drug found in the supernatant.

The encapsulation efficiency (EE) of the liposomes was determined using the equation described above. The maximal EE for BTZ and Y27632 was determined to be 77% and 55%, respectively.

### Example 11: Effect of Free and Liposomal Y27632 on Trans-Endothelial Migration of MM Cells In Vitro

To evaluate the effect of Y27632 administration in free and liposomal forms on trans-endothelial migration of in vitro MM cells, the following experiments were conducted.

Trans-endothelial migration was by incubating HUVECs (5×10³ cells) overnight in the upper chamber of a Boyden chamber (Corning), followed by an adhesion assay. MM.1S cells were pre-treated with (or without) free Y27632 (25 μM) or liposomal Y27632 (25 μM equivalent) for 3 hours. The pre-treated MM.1 S cells were then placed in the upper migration chamber in the presence or absence of 30 nM SDF-1 in the lower chamber. After 3 hours of incubation, MM.1 S cells that migrated to the lower chambers were counted by flow cytometry. The in vitro data were expressed as means±standard deviation. Results were analyzed using a student t-test or ANOVA for statistical significance and were considered significantly different for all p-values below 0.05.

As illustrated in FIG. 4C, both free and liposomal Y27632 reversed the SDF-induced trans-endothelial migration of MM cells in vitro.

### Example 12: Effect of Free and Liposomal Y27632 on Mobilization of MM Cells to the Circulation In Vivo

To evaluate the effect of Y27632 administration in free and liposomal forms on the migration of MM cells to circulation in vivo, the following experiments were conducted.

The mouse model described in Example 2 was used for these experiments. MM.1S-GFP-Luc cells (2×10⁶ cells/mouse) were injected intravenously into nine mice, and tumor progression was confirmed using BLI at 4 weeks post-injection. Mice were then treated with intravenous injections of i) free Y27632 (2.5 mg/kg, n=3); ii) Y27632-loaded non-targeted liposomes (2.5 mg/kg equivalent, n=3); and iii) Y27632-loaded PSGL-1-targeted liposomes (2.5 mg/kg, n=3). Blood was collected from the tail vein of each mouse before injection, 2 after injection, and 4 hours after injection. All blood samples were lysed with 1× red blood cell lysis buffer (BioLegend, San Diego, Calif.) using the manufacturer's instructions and the peripheral blood mononuclear cells (PBMCs) were analyzed via flow cytometry. The in vitro data were expressed as means±standard deviation. Results were analyzed using a student t-test or ANOVA for statistical significance and were considered significantly different for all p-values below 0.05.

The results of these in vivo experiments are summarized in FIG. 4D. The administration of Y27632 in either free administration or non-targeted liposomal form resulted in a similar mobilization of MM cells to the circulation. By contrast, the administration of Y27632 in the PSGL-1-targeted liposomal form induced significantly more mobilization of MM cells to the circulation, indicating a more profound inhibition of the interaction of MM cells and the BMME.

### Example 13: Effect of Free and Liposomal Drugs on Cell Signaling in MM Cells and ECs

To compare the effect of the in vitro administration of Y27632 in free and liposomal formats on adhesion signaling in MM cells and HUVECs, the following experiments were conducted.

Monoclonal antibodies (mAb) used for western blot were purchased from Cell Signaling Technology (Danvers, Mass.). Phospho-Akt (pAKT; #4060), phospho-Erk1/2 (pERK; #4370), phospho-Rb (pRB; #9308), p21 (#2947), cleaved Caspase3 (cCasp3; #9664), cleaved Caspase 9 (cCasp9; #7237), cleaved PARP (cPARP; #5625), phospho-FAK (pFAK; #3284), phospho-SRC (pSRC; #6943), and phosphor-S6 ribosomal protein (pS6R; #4858) were used at a dilution of 1:1000. α-Tubulin (#2125) was used as a loading control at a dilution of 1:3000. The immunoblots were detected using an ECL Plus chemiluminescent system (Perkin Elmer, Waltham, Mass.).

HUVECs and MM1.S cells were co-cultured overnight and treated with vehicle (control), free Y27632 (25 μM), free BTZ (5 nM), empty liposomes, liposomal Y27632 (25 μM), liposomal BTZ (5 nM) for 6 hours. MM cells were then separated from the HUVECs and both cell types were collected. The proteins were then extracted and subjected to immunoblotting for pSRC, pFAK, p21, pRB, cPARP, cCasp3, cCasp9, pAKT, pS6R, pERK, and α-Tubulin.

The immunoblotting protocol was performed as previously described below. Briefly, cells were lysed with 1× lysis buffer (Cell Signaling, #9803), the protein concentration was determined by Bradford assay (BioRad, Hercules, Calif.), and 50 μg of protein was loaded per lane. Electrophoresis was performed using NuPAGE 4%-12% Bis-Tris gels (Novex, Life Technologies, Grand Island, N.Y.) and transferred to a nitrocellulose membrane using iBlot (Invitrogen). Membranes were blocked with 5% non-fat milk in Tris-Buffered Saline/Tween20 (TBST) buffer and incubated with primary antibodies overnight at 4° C. The membranes were then washed with TBST for 30 minutes, incubated for 1 hour at room temperature with horseradish peroxidase (HRP)-conjugated secondary antibody, washed, and developed using Novex ECL Plus chemiluminescent Kit. Images were taken using a ChemiDoc XRS imaging system (Bio-Rad).

The immunoblotted adhesion signaling proteins from lysed MMs cultured with various treatments are summarized in FIG. 4A and the immunoblotted adhesion signaling proteins from lysed HUVECs cultured with various treatments are summarized in FIG. 4B. Treatment with empty liposomes did not induce any change in adhesion signaling proteins relative to the untreated cells for either MMs or HUVECs. Further, treatment using free Y27632 induced decreased adhesion signaling relative to the untreated cells for either MMs or HUVECs. Treatment of both MMS and HUVECs with liposomal Y27632 decreased adhesion signaling proteins (pSRC and pFAK) and had a greater effect than the administration of Y27632 in free form for both MM and HUVECs.

The results of these experiments found that liposomal Y27632 downregulated adhesion signaling (SRC and FAK) in MM and BMME cells in vitro, and the effect of liposomal Y27632 administration was comparable to or more profound than the effect of free Y27632 administration.

### Example 14: Effect of Free and Liposomal BTZ on MM and EC Viability In Vitro

To compare the effect of free and liposomal BTZ administration on apoptosis and proliferation signaling by in vivo MM cells and ECs, the following experiments were conducted.

MM cells were co-cultured overnight and treated with vehicle (untreated control), free BTZ (5 nM), empty liposomes, and liposomal BTZ (5 nM) for 6 hours. MM cells were collected and cell proteins were subsequently extracted and subjected to immunoblotting for pSRC, pFAK, p21, pRB, cPARP, cCasp3, cCasp9, pAKT, pS6R, pERK, and α-Tubulin as described in Example 13.

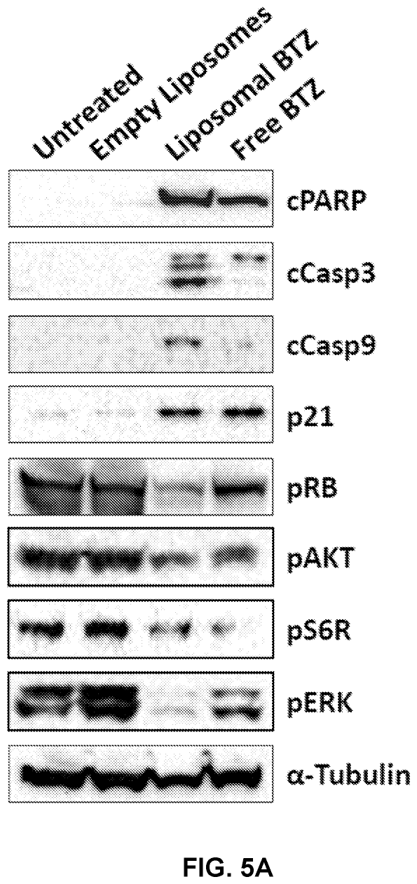

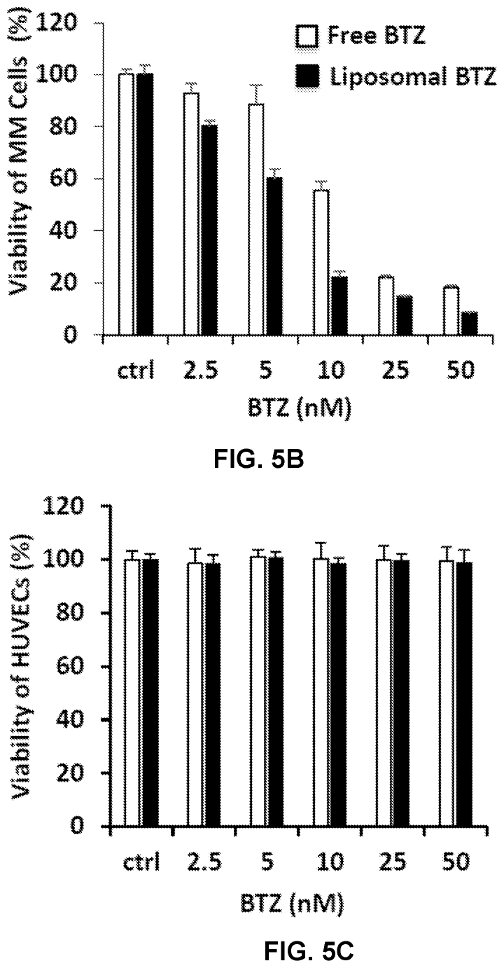

FIG. 5A summarizes the results of these experiments. Empty liposomes did not have any effect on apoptosis and proliferation signaling. However, liposomal BTZ increased pro-apoptotic signaling (cPARP, cCasp3, and cCasp9) and decreased proliferation signaling (pRb, pAKT, pS6R, and pERK) more profoundly compared to free BTZ.

To compare the effect of free and liposomal BTZ administration on the viability of in vivo MM cells and HUVECs, the following experiments were conducted

DiD-labeled HUVECs and DiO-labeled MM.1S cells were co-cultured overnight and treated with free BTZ (0-50 nM) or liposomal BTZ (0-50 nM-equivalent) for 24 hours, and the survival of the MMs and HUVECs was analyzed via flow cytometry. MM cells were gated as DiO+ cells and HUVECs were gated as DiD+ cells, and each cell population was counted and normalized against counting beads (Invitrogen), and survival was calculated as a percentage of vehicle-treated controls. The in vitro data were expressed as means±standard deviation. Results were analyzed using a student t-test or ANOVA for statistical significance and were considered significantly different for all p-values below 0.05.

As illustrated in FIG. 5B, liposomal BTZ killing of MM cells was more effective than the free counterpart in vitro with IC50 values of approximately 5 nM and 10 nM, respectively. No effect of BTZ was detected on the survival of HUVECs in either free or liposomal form, as summarized in FIG. 5C.

The results of these experiments found that liposomal BTZ downregulated proliferation signaling and increased apoptosis signaling in MM cells. Liposomal BTZ also induced cytotoxicity to MM cells, but not ECs in vitro. The effect of the liposomal BTZ was similar to or more profound than free BTZ (FIGS. 5B and 5C).

### Example 15: Efficacy of BTZ and Y27632-Loaded PSGL-1-Targeted Liposomes on MM Tumor Progression In Vivo

To assess the efficacy of BTZ and Y27632-loaded PSGL-1-targeted liposomes on MM tumor progression in vivo, the following experiments were conducted.

The mouse model described in Example 2 was used for these experiments. MM.1S-GFP-Luc cells (2×10⁶ cells/mouse) were injected intravenously into 84 mice, and tumor progression was confirmed using BLI at 3 weeks post-injection. Mice were randomized into 12 groups of 7 mice each, which received weekly intravenous injections of: (i) saline, (ii) Y27632 as a free drug (2.5 mg/kg), (iii) BTZ as a free drug (1 mg/kg), (iv) combination of free BTZ and free Y27632, (v) empty non-targeted liposomes, (vi) non-targeted liposomal Y27632 (2.5 mg/kg-equivalent), (vii) non-targeted liposomal BTZ (1 mg/kg), (viii) non-targeted liposomal combination of BTZ and Y27632 in the same liposome (2.5 mg/kg and 1 mg/kg, respectively), (ix) empty PSGL-1-targeted liposomes, (x) PSGL-1-targeted liposomal Y27632 (2.5 mg/kg), (xi) PSGL-1-targeted liposomal BTZ (1 mg/kg), (xii) PSGL-1-targeted liposomal combination of BTZ and Y27632 in the same liposome (2.5 mg/kg and 1 mg/kg, respectively). Tumor progression was assessed weekly by BLI, weight was recorded twice a week, and survival and general health of mice were recorded daily. The in vivo data were expressed as means±standard deviation. Results were analyzed using a student t-test or ANOVA for statistical significance and were considered significantly different for all p-values below 0.05.

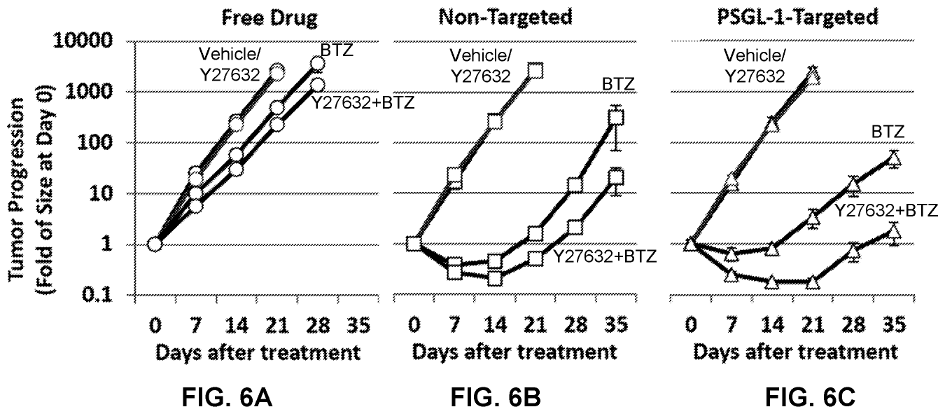

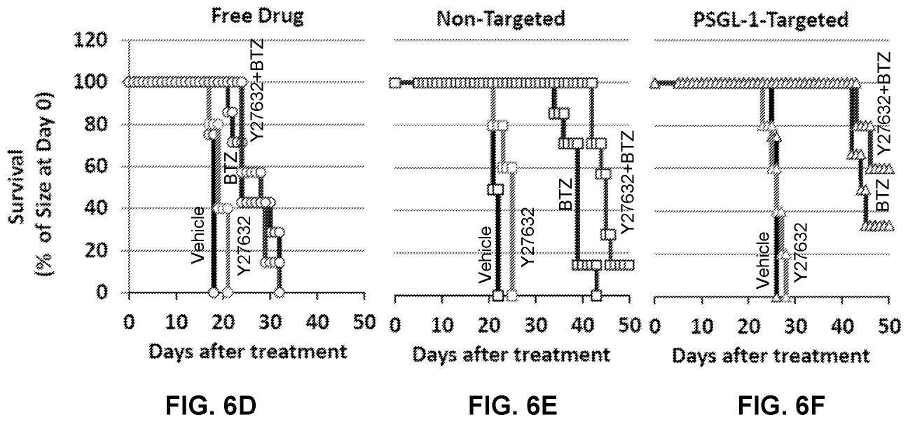

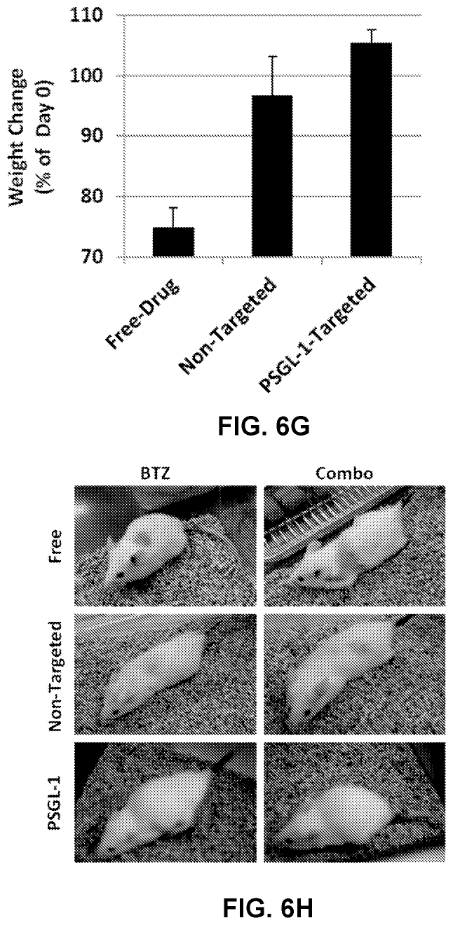

Free BTZ delayed significantly the tumor growth, and the combination of Y27632 and BTZ significantly improved the effect of BTZ (FIG. 6A). Non-targeted BTZ-loaded liposomes dramatically reduced the tumor progression about 3 orders of magnitude compared to the non-targeted empty liposomes; non-targeted multi-drug (BTZ and Y27632) liposomes improved the effect of BTZ-loaded liposomes (FIG. 6B). PSGL-1-targeted BTZ-loaded liposomes dramatically reduced the tumor progression about 3 orders of magnitude compared to non-targeted empty liposomes; PSGL-1-targeted multi-drug liposomes improved the effect of BTZ and reduced tumor progression by an order of magnitude compared to the PSGL-1-targeted BTZ-loaded liposomes (FIG. 6C).

With respect to survival, free BTZ significantly prolonged the survival of the mice; however, there was not an improvement in survival when combined with Y27632 (FIG. 6D). The mice treated with non-targeted BTZ-loaded liposomes lived significantly longer than the vehicle and Y27632-loaded liposomes, and the non-targeted multi-drug liposomes improved the survival rate from 0 to 14% past 50 days compared to the BTZ-only liposomes (FIG. 6E). PSGL-1-targeted BTZ-loaded liposomes significantly extended the survival of the mice compared to the PSGL-1-targeted empty and Y27632-loaded liposomes, and the PSGL-1-targeted multi-drug liposomes doubled the survival rate from ˜30 to 60% compared to the PSGL-1-targeted BTZ-only liposomes (FIG. 6F).

The side effects observed in each of the treatment groups were also monitored. As expected, the free drug treatments induced significant weight loss, whereas the non-targeted and PSGL-1-targeted treatments significantly reduced the weight loss seen in the free drug regimen. In addition, the weights of the mice treated with the PSGL-1-targeted treatments increased by about 5% compared to the weights measured prior to therapy (FIG. 6G). The non-targeted form of BTZ and combination treatment improved the effect of hair loss compared to the free drug, and with the use of PSGL-1-targeted liposomes, absolutely no hair loss was seen for the mice (FIG. 6H).

The results of these experiments found that the concept of combining chemotherapy with a BMME-disrupting agent as an approach to sensitize MM to therapy and overcome BMME-induced drug resistance was eminent in all three forms of delivery (free drugs, non-targeted liposomes, and PSGL-1 targeted liposomes). The combination of BTZ with Y27632 resulted in better tumor efficacy and survival than BTZ alone (FIGS. 6A, 6B, 6C, 6D, 6E, and 6F). Further, the delivery of the treatment with liposomes improved the therapeutic efficacy of BTZ alone or in combination with Y27632, compared to administering as free drugs; this is most likely due to specific accumulation in the tumor due to the EPR effect of liposomes in general. Targeting with PSGL-1, on the other hand, improved the specificity and therapeutic efficacy of BTZ alone or in combination with Y27632 even more due to the specific interaction with the MM-associated endothelium. The combination effect of BTZ and Y27632 was more profound in the liposomal formulations compared to administering as free drugs due to synchronized delivery, and this effect was more pronounced in the PSGL-1-targeted liposomes (FIGS. 6B, 6C, 6E, and 6F). Furthermore, the PSGL-1-targeted liposomes reduced the side effects of BTZ, in which these liposomes did not cause weight loss or hair loss compared to the non-targeted liposomes and the free drugs (FIG. 6H).

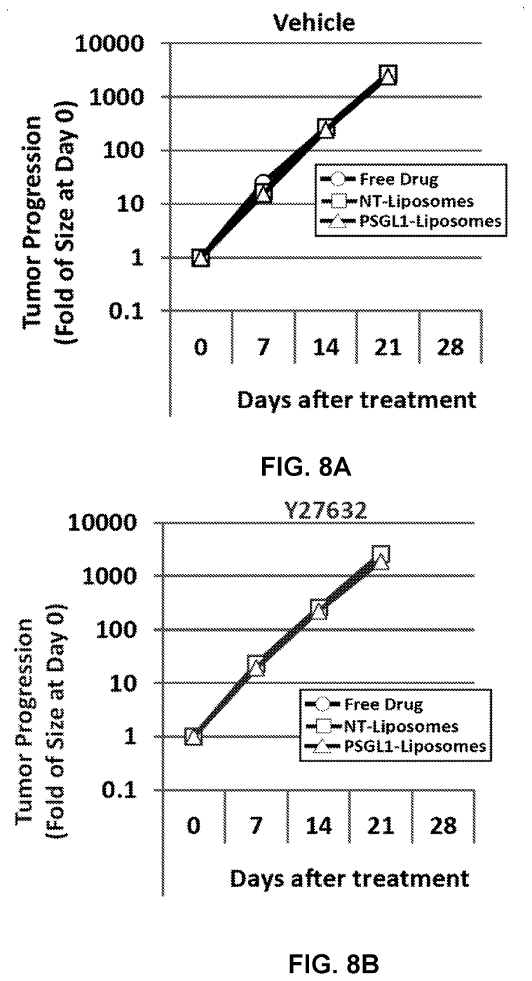

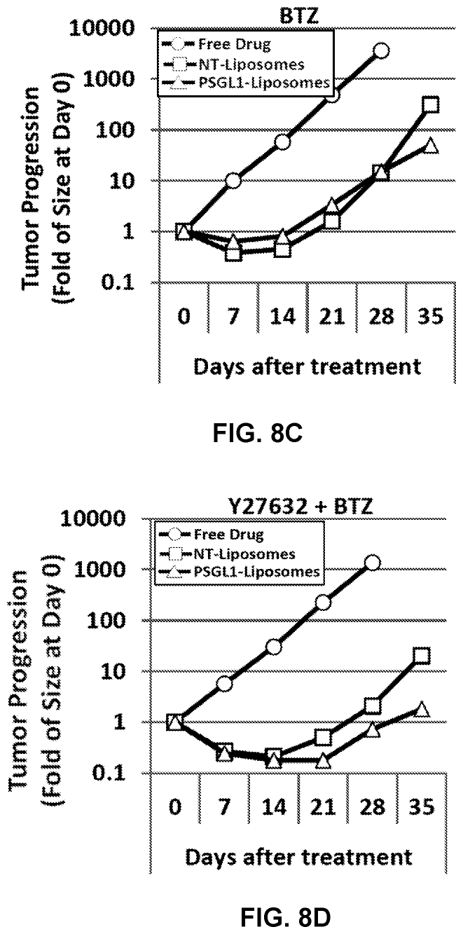

The groups treated with non-targeted empty liposomes and the PSGL-1-targeted empty liposomes showed tumor growth rate similar to the vehicle-treated control (FIG. 8A). Moreover, Y27632 alone administered as a free drug, loaded onto non-targeted liposomes, or loaded onto PSGL-1-targeted liposomes did not affect the tumor growth (FIG. 8B). The two liposomal BTZ (alone) formulations showed a profound decrease in tumor progression compared to free BTZ with a slight advantage for the PSGL-1-targeted liposomes (FIG. 8C). The combination of BTZ and Y27632 showed more efficacy than BTZ alone in all formulations (free, non-targeted and PSGL-1-targeted), while the most efficacious effect was observed in the group treated with PSGL-1-targeted multidrug liposomes compared to the other 11 treatment groups (FIG. 8D).

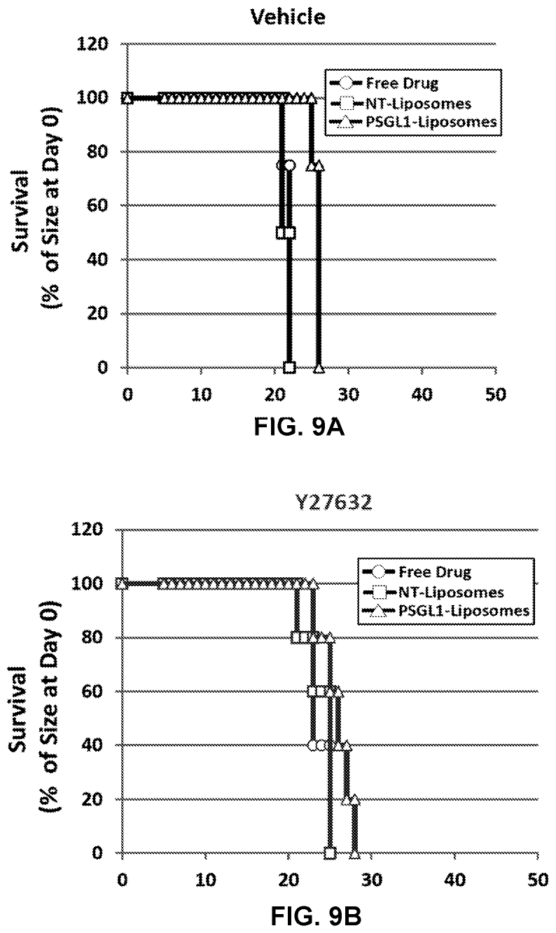

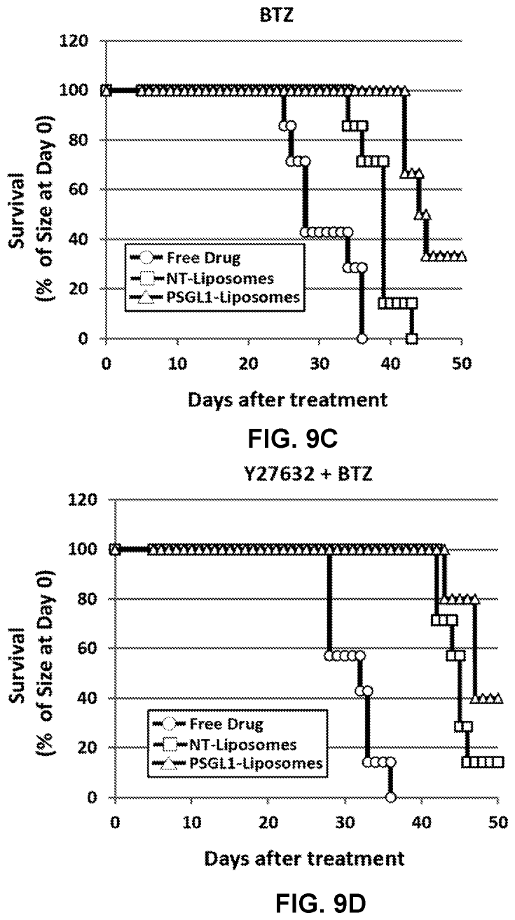

With regards to survival, the groups treated with non-targeted empty liposomes and the PSGL-1-targeted empty liposomes died around the same time as the vehicle-treated controls (FIG. 9A). In addition, Y27632 alone as free drugs, in non-targeted liposomes or PSGL-1-targeted liposomes did not extend the survival of the MM-bearing mice (FIG. 9B). The non-targeted BTZ-loaded liposomes extended the survival of the MM-bearing mice and the PSGL-1-targeted BTZ liposomes improved the survival of mice compared to the non-targeted BTZ-loaded liposomes (FIG. 9C). The combination of BTZ and Y27632 prolonged the survival of mice compared to BTZ alone in all formulations (free, non-targeted, and PSGL-1-targeted). Moreover, the PSGL-1-targeted multidrug liposomes prolonged the survival of 60% of the mice, having a more favorable result compared to the other 11 treatment groups (FIG. 9D).

What is claimed is:

1. A composition for treating multiple myeloma (MM) within a patient in need, the composition comprising:
a. a liposome comprising a lipid bilayer shell forming an outer surface and an inner surface enclosing a fluid-filled center;
b. a targeting moiety coupled to the outer surface;
c. a treatment compound disposed within the lipid bilayer shell or within the fluid-filled center; and
d. an efficacy-enhancing compound disposed within the lipid bilayer shell or within the fluid-filled center.

2. The composition of claim 1, wherein the targeting moiety comprises PSGL-1.

3. The composition of claim 1, wherein the treatment compound comprises a proteasome-inhibiting compound.

4. The composition of claim 3, wherein the proteasome-inhibiting compound is bortezomib, the bortezomib disposed within the lipid bilayer shell between the inner and outer surfaces.

5. The composition of claim 4, wherein the bortezomib is disposed within the lipid bilayer shell at an encapsulation efficiency ranging from about 70% to about 80%.

6. (canceled)

7. The composition of claim 1, wherein the efficacy-enhancing compound is a BMME-disrupting agent selected from a CXCR4 inhibitor and a ROCK inhibitor.

8. The composition of claim 7, wherein the efficacy-enhancing compound is the ROCK inhibitor, the ROCK inhibitor comprising Y27632, the Y27632 disposed within the fluid-filled center.

9. (canceled)

10. The composition of claim 8, wherein the Y27632 is disposed within the fluid-filled center at an encapsulation efficiency ranging from about 40% to about 60%.

11.-14. (canceled)

15. The composition of claim 1, wherein the liposome further comprises an average size ranging from about 125 nm to about 175 nm.

16. (canceled)

17. The composition of claim 1, wherein the zeta potential of the liposomes is at least about 28 mV.

18. The composition of claim 1, wherein the liposome further comprises DPPC, Chol, DSPE-mPEG2000, and DSPE-PEG(2000)-succinyl, and any combination thereof.

19. The composition of claim 18, wherein the liposome comprises DPPC, Chol, DSPE-mPEG2000, and DSPE-PEG(2000)-succinyl at molar ratio of 6:3:0.5:0.5 (DPPC:Chol:DSPE-mPEG2000:DSPE-PEG(2000)-succinyl).

20. The composition of claim 1, wherein the fluid-filled center comprises a hydrophilic fluid.

21. The composition of claim 1, further comprising a liquid carrier, wherein the liposomes are suspended within the liquid carrier.

22. The composition of claim 21, wherein the liposomes are suspended within the liquid carrier at a concentration of 2 mg of liposomes per mL of lipids.

23. A method of specifically delivering a therapeutic composition of PSGL-1 functionalized liposomes loaded with bortezomib and Y27632 to tumor cells of a subject, the method comprising administering an effective amount of the therapeutic composition to the subject, wherein the liposomes comprise:
a. a lipid bilayer shell forming an outer surface and an inner surface enclosing a fluid-filled center;
b. the PSGL-1 coupled to the outer surface;
c. the bortezomib disposed within the lipid bilayer shell between the inner and outer surfaces; and
d. the Y27632 disposed within the fluid-filled center.

24. The method of claim 23, wherein the therapeutic composition is administered by injection or infusion.

25. The method of claim 24, wherein the therapeutic composition is injected or infused at a dose of about 2.5 mg/kg of BTZ and about 2.5 mg/kg of Y27632.

26. The method of claim 23, wherein the bortezomib is disposed within the lipid bilayer shell at an encapsulation efficiency ranging from about 70% to about 80% and the Y27632 is disposed within the fluid-filled center at an encapsulation efficiency ranging from about 40% to about 60%.

27. The method of claim 23, wherein the therapeutic composition further comprises a liquid carrier, wherein the liposomes are suspended within the liquid carrier at a concentration of 2 mg of liposomes per mL of lipids.
